(19) United States
(12) Patent Application Publication
(10) Pub. No.: US 2023/0389418 A1
(43) Pub. Date: Nov. 30, 2023

(54) MATERIAL OF PHOTOELECTRIC CONVERSION ELEMENT FOR IMAGING, AND PHOTOELECTRIC CONVERSION ELEMENT

(71) Applicant: NIPPON STEEL Chemical & Material Co., Ltd., Tokyo (JP)

(72) Inventors: Atsushi KAWADA, Tokyo (JP); Munetomo INOUE, Tokyo (JP); Kentaro HAYASHI, Tokyo (JP)

(73) Assignee: NIPPON STEEL Chemical & Material Co., Ltd., Tokyo (JP)

(21) Appl. No.: 18/031,951

(22) PCT Filed: Nov. 25, 2021
(86) PCT No.: PCT/JP2021/043221

§ 371 Date: Apr. 14, 2023

(30) Foreign Application Priority Data
Nov. 27, 2020 (JP) 2020-196805

(51) Int. Cl. H10K 85/60; H10K 50/15; H10K 50/16

(52) U.S. Cl. CPC H10K 85/6572; H10K 85/6574; H10K 85/6576; H10K 85/615; H10K 85/622; H10K 50/15; H10K 50/16; H10K 2101/30

(57) ABSTRACT

Provided are a material that achieves higher sensitivity and higher resolution of a photoelectric conversion element for imaging, and a photoelectric conversion element for imaging using the above material. The material for a photoelectric conversion element for imaging includes an indolocarbazole compound having a pentacyclic fused-ring structure having two heteroatoms, or an analogue compound thereof. The material is a compound having, as a group bonded to a nitrogen atom or a heteroatom, an alkyl group, a substituted or non-substituted aromatic hydrocarbon group, a substituted or non-substituted π-electron excess heteroaromatic group, or a linked aromatic group formed by linking of two to six of these aromatic groups.

### TECHNICAL FIELD

The present invention relates to a material for a photoelectric conversion element for imaging and a photoelectric conversion element for imaging using the same.

In recent years, development of an organic electronic device using a thin film formed with an organic semiconductor (also referred to as an organic charge transport material) is in progress. Examples thereof include an electroluminescent element, a solar cell, a transistor element, and a photoelectric conversion element. In particular, development of an organic EL element, which is an electroluminescent element with an organic substance, is most advanced among them. The applications for smartphones, TV and the like are in progress, and development for a purpose of further higher functionality is continuously conducted.

On the photoelectric conversion element, an element using a P-N junction of an inorganic semiconductor, such as silicon, has been conventionally developed and practically used, and made are investigations for high functionalization of a digital camera and a camera for a smartphone and investigation for application for a monitoring camera, a sensor for an automobile, and the like. However, problems for these various uses include improving sensitivity and micronizing a pixel (improving resolution). For the photoelectric conversion element using an inorganic semiconductor, a mainly adopted method for obtaining a color image is disposing color filters corresponding to RGB, which are the three primary colors of light, on a light receiving part of the photoelectric conversion element. This method has problems in terms of utilization efficiency of an incident light and resolution, because the method disposes the RGB color filters on a plane (Non Patent Literature 1 and 2).

As a solution for such problems of the photoelectric conversion element, a photoelectric conversion element using an organic semiconductor instead of the inorganic semiconductor is developed (Non Patent Literature 1 and 2). This utilizes “an ability to selectively absorb only light having a specific wavelength region with high sensitivity” that the organic semiconductor has, and proposed is stacking photoelectric conversion elements composed of organic semiconductors corresponding to the three primary colors of light to solve the problem of improving the sensitivity and improving the resolution. An element in which a photoelectric conversion element composed of the organic semiconductor and a photoelectric conversion element composed of the inorganic semiconductor are stacked is also proposed (Non Patent Literature 3).

Here, the photoelectric conversion element composed of the organic semiconductor is an element in which a photoelectric conversion layer composed of a thin film of the organic semiconductor is disposed between two electrodes, and as necessary, a hole blocking layer and/or an electron blocking layer is disposed between the photoelectric conversion layer and the two electrodes. In the element, light having a desired wavelength is absorbed in the photoelectric conversion layer to generate an exciter, and then charge separation of the exciter generates a hole and an electron. Thereafter, the hole and the electron move toward each electrode to convert the light into an electric signal. For a purpose of accelerating this process, a method of applying a bias voltage between both the electrodes is commonly used, but one of objects is reducing a leakage current from both the electrodes generated by applying the bias voltage. Accordingly, it can be mentioned that controlling the move of the hole and the electron in the photoelectric conversion element is a key to exhibit characteristic of the photoelectric conversion element.

The organic semiconductor used for each layer of the photoelectric conversion element can be classified into a P-type organic semiconductor and an N-type organic semiconductor. The P-type organic semiconductor is used as a hole transport material, and the N-type organic semiconductor is used as an electron transport material. To control the move of the hole and the electron in the aforementioned photoelectric conversion element, made are various developments of an organic semiconductor having appropriate physical properties such as hole mobility, electron mobility, an energy value of a highest occupied molecular orbital (HOMO), and an energy value of a lowest unoccupied molecular orbital (LUNG). However, the organic semiconductor still has insufficient characteristics, and has not been utilized in commercial practice.

Patent literature 1 proposes an element using quinacridone as the P-type organic semiconductor and subphthalocyanine chloride as the N-type organic semiconductor for the photoelectric conversion layer, and an indolocarbazole derivative for a first buffer layer (which has presumably the same means as the electron blocking layer) disposed between the photoelectric conversion layer and the electrode. The application of the indolocarbazole derivative therein is limited to the first buffer layer, and applicability for the photoelectric conversion layer is unknown.

Patent literature 2 proposes an element using, for the photoelectric conversion layer, a chrysenodithiophene derivative as the P-type organic semiconductor and fullerenes or a subphthalocyanine derivative as the N-type organic semiconductor.

Patent literature 3 proposes an element using a benzodifuran derivative for the electron blocking layer disposed between the photoelectric conversion layer and the electrode.

However, further higher sensitivity and higher resolution have been desired for the photoelectric conversion element for imaging.

### CITATION LIST

### Patent Literature

Patent Literature 1
JP 2018-85427(A)

Patent Literature 2
JP 2019-54228(A)

Patent Literature 3
JP 2019-57704(A)

### Non Patent Literature

Non Patent Literature 1
NHK Science & Technology Research Laboratories R & D No. 132, 2012.3, pp. 4-11

Non Patent Literature 2
NHK Science & Technology Research Laboratories R & D No. 174, 2019.3, pp. 4-17

Non Patent Literature 3
2019 IEEE International Electron Devices Meeting (IEDM), pp. 16.6.1-16.6.4 (2019)

### SUMMARY OF INVENTION

In the use of the photoelectric conversion element for imaging for highly functionalizing a digital camera and a camera for a smartphone and for application for a monitoring camera, a sensor for an automobile, and the like, challenges are further higher sensitivity and higher resolution. In view of such a circumstance, an object of the present invention is to provide a material that achieves higher sensitivity and higher resolution of the photoelectric conversion element for imaging, and a photoelectric conversion element for imaging using the same.

The present inventors have made intensive investigation, and consequently found that using an indolocarbazole derivative as a hole transport material efficiently proceeds a process of generating a hole and an electron by charge separation of an exciter in a photoelectric conversion layer, and control of moving of the hole and the electron in the photoelectric conversion element. This finding has led to the completion of the present invention.

Specifically, the present invention relates to a material for a photoelectric conversion element for imaging having a structure of the following general formula (1) or (2).

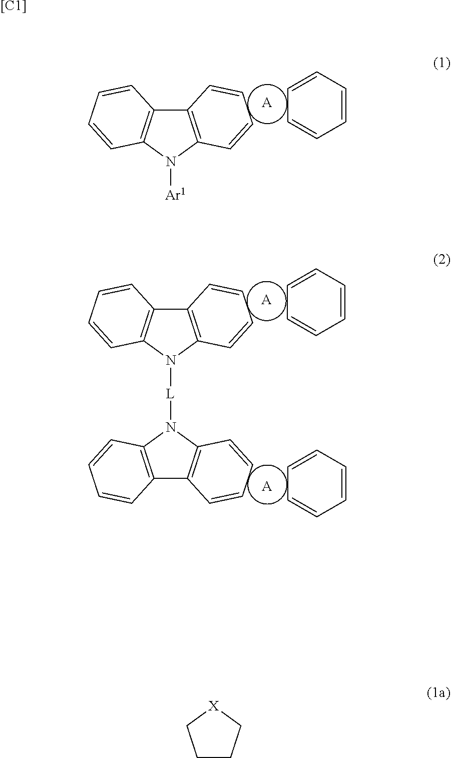

In the formulae (1) and (2), the ring A independently represents a heterocyclic ring represented by the formula (1a) and fused with an adjacent ring at any position.

X represents O, S, or N—Ar².

Ar¹ and Ar² each independently represent an alkyl group having 1 to 20 carbon atoms, a substituted or non-substituted aromatic hydrocarbon group having 6 to 30 carbon atoms, a substituted or non-substituted π-electron excess heteroaromatic group having 4 to 30 carbon atoms, or a substituted or non-substituted linked aromatic group formed by linking of two to six aromatic groups selected from the aromatic hydrocarbon group and the π-electron excess heteroaromatic group. When Ar¹ and Ar² are linked aromatic groups formed only by the aromatic hydrocarbon group, Ar¹ and Ar² are not simultaneously biphenyl groups.

L represents a divalent substituted or non-substituted aromatic hydrocarbon group having 6 to 30 carbon atoms, a substituted or non-substituted π-electron excess heteroaromatic group having 4 to 30 carbon atoms, or a linked aromatic group formed by linking of two to six aromatic rings of aromatic groups selected from the aromatic hydrocarbon group and the π-electron excess heteroaromatic group.

In a preferable aspect, at least one of the Ar¹ or Ar² contains at least one substituted or non-substituted tricyclic fused skeleton. The tricyclic skeleton is preferably a substituted or non-substituted carbazole, dibenzofuran, or dibenzothiophene skeleton, and further preferably a substituted or non-substituted carbazole skeleton.

In the material for a photoelectric conversion element, an energy level of highest occupied molecular orbital (HOMO) obtained by structural optimization calculation with a density functional calculation B3LYP/6-31G(d) is preferably −4.5 eV or lower, or an energy level of lowest unoccupied molecular orbital (LUNO) is preferably −2.5 eV or higher.

In addition, the material for a photoelectric conversion element preferably has a hole mobility of 1×10⁻⁶ cm²/Vs or more, or is preferably amorphous.

The above material for a photoelectric conversion element may be used as a hole transport material for a photoelectric conversion element for imaging.

In addition, the present invention relates to a photoelectric conversion element for imaging, comprising a photoelectric conversion layer and an electron blocking layer between two electrodes, wherein at least one layer of the photoelectric conversion layer or the electron blocking layer contains the above material for a photoelectric conversion element.

In the photoelectric conversion element of the present invention, the photoelectric conversion layer may contain an electron transport material, and the electron blocking layer may contain the above material for a photoelectric conversion element.

Using the material for a photoelectric conversion element of the present invention can achieve appropriate move of the hole and the electron in the photoelectric conversion element for imaging, and consequently enables to reduce a leakage current generated by applying a bias voltage during the conversion of light into electric energy. As a result, a photoelectric conversion element that achieves a low dark current value and a high contrast ratio can be obtained.

### BRIEF DESCRIPTION OF DRAWING

FIG. 1 is a sectional schematic view illustrating a structure example of a photoelectric conversion element for imaging.

### DESCRIPTION OF EMBODIMENTS

The photoelectric conversion element of the present invention has at least one organic layer between two electrodes. This organic layer contains the material for a photoelectric conversion element for imaging represented by the general formula (1) or (2) (also referred to as the material for a photoelectric conversion element or the material of the present invention). As necessary, the photoelectric conversion element can have a plurality of the organic layers containing the material for a photoelectric conversion element.

The general formulae (1) and (2) will be described. In the general formulae (1) and (2), a common symbol has a same mean.

The ring A represents a heterocyclic ring represented by the formula (la) and fused with an adjacent ring at any position.

In the formula (1a), X represents O, S, or N—Ar², and preferably N—Ar². When X is N—Ar², the pentacyclic fused ring in the general formula (1) represents an indolocarbazole skeleton, and there are five isomers represented by the following formulae (V), (W), (X), (Y), and (Z). The indolocarbazole skeleton is preferably the formula (V), (W), or (Y). Note that, when X is O or S, there are also isomers similar to the indolocarbazole skeleton.

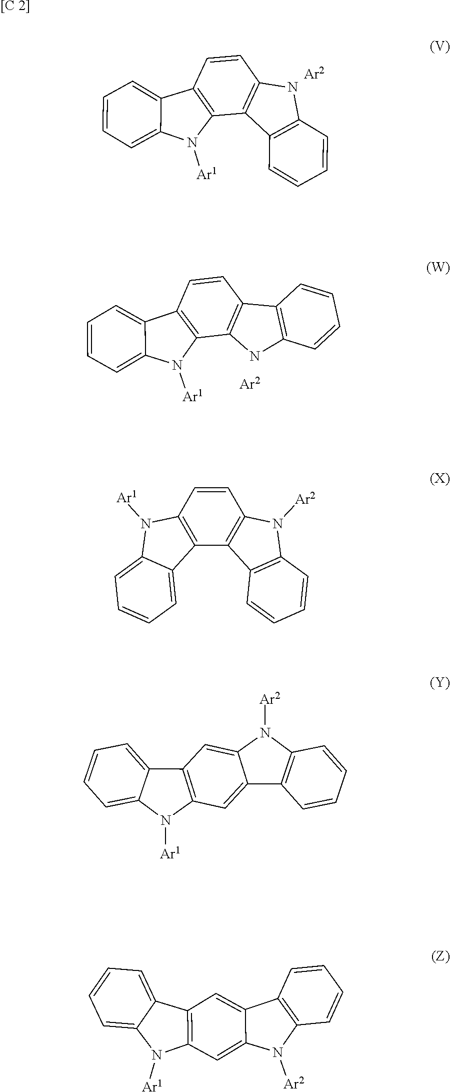

In the general formula (2), there are two pentacyclic fused rings and two rings A. When all X in the formula (1a) is N—Ar², the pentacyclic fused rings become indolocarbazole skeletons, and there are isomers similar to the above. A linking form of these indolocarbazole skeletons has a combination of same isomers and a combination of different isomers, and preferably the combination of same isomers. Note that, when X is O or S, there are also isomers similar to the indolocarbazole skeleton. Although there is a combination of different isomers, a combination of same isomers is preferable.

Examples of the combination of same isomers include the following formulae (21) to (27). Among them, the formula (21), (23), or (24) is preferable.

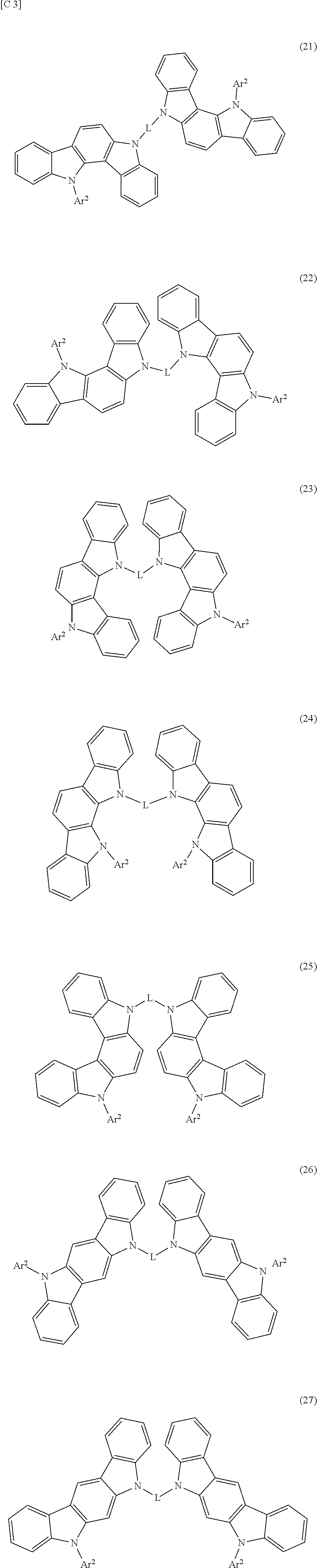

Examples of the combination of different isomers are shown below, but the combination is not limited thereto.

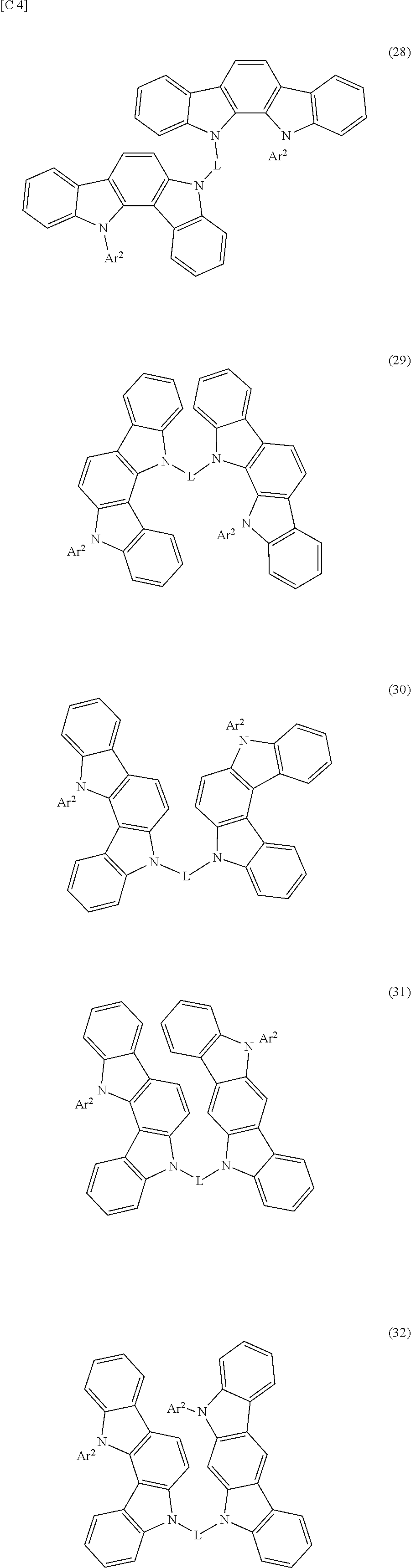

Ar¹ and Ar² are independently an alkyl group having 1 to 20 carbon atoms, a substituted or non-substituted aromatic hydrocarbon group having 6 to 30 carbon atoms, a substituted or non-substituted π-electron excess heteroaromatic group having 4 to 30 carbon atoms, or a substituted or non-substituted linked aromatic group formed by linking of two to six aromatic rings of aromatic groups selected from the aromatic hydrocarbon group and the π-electron excess heteroaromatic group. Ar¹ and Ar² are preferably a substituted or non-substituted aromatic hydrocarbon group having 6 to 20 carbon atoms, a substituted or non-substituted π-electron excess heteroaromatic group having 4 to 20 carbon atoms, or a substituted or non-substituted linked aromatic group formed by linking of two to four aromatic rings of aromatic groups selected from the aromatic hydrocarbon group and the π-electron excess heteroaromatic group. Ar¹ and Ar² are further preferably a substituted or non-substituted aromatic hydrocarbon group having 6 to 14 carbon atoms, a substituted or non-substituted π-electron excess heteroaromatic group having 4 to 14 carbon atoms, or a substituted or non-substituted linked aromatic group formed by linking of two to four aromatic rings of aromatic groups selected from the aromatic hydrocarbon group and the π-electron excess heteroaromatic group.

At least one of Ar¹ and Ar² is also preferably the π-electron excess heteroaromatic group or a substituted or non-substituted linked aromatic group containing at least one of the π-electron excess heteroaromatic group.

When Ar¹ and Ar² are linked aromatic groups formed only by the aromatic hydrocarbon groups, Ar¹ and Ar² are preferably groups different from each other, and Ar¹ and Ar² are not simultaneously biphenyl groups. When the general formula (1) is the formula (V), Ar¹ and Ar² being linked aromatic groups formed only by the hydrocarbon aromatic group are preferably different groups.

In the general formula (2), L represents a divalent substituted or non-substituted aromatic hydrocarbon group having 6 to 30 carbon atoms, a substituted or non-substituted π-electron excess heteroaromatic group having 4 to 30 carbon atoms, or a linked aromatic group formed by linking of two to six aromatic rings of aromatic groups selected from the aromatic hydrocarbon group and the π-electron excess heteroaromatic group. L is preferably a divalent substituted or non-substituted aromatic hydrocarbon group having 6 to 14 carbon atoms, a substituted or non-substituted π-electron excess heteroaromatic group having 4 to 14 carbon atoms, or a linked aromatic group formed by linking of two to three aromatic rings of aromatic groups selected from the aromatic hydrocarbon group and the π-electron excess heteroaromatic group.

When Ar¹ and Ar² are an alkyl group having 1 to 20 carbon atoms, the alkyl group having 1 to 20 carbon atoms may be any of linear, branched, and cyclic alkyl groups. Examples thereof include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, a 4-butylcyclohexyl group, and a 4-dodecylcyclohexyl group. Preferable examples thereof include a linear, branched, or cyclic alkyl group having 1 to 10 carbon atoms.

Example of the non-substituted aromatic hydrocarbon group of Ar¹ or Ar² having 6 to 30 carbon atoms include: monocyclic hydrocarbon aromatic group, such as benzene; bicyclic aromatic hydrocarbons, such as naphthalene; tricyclic aromatic hydrocarbons, such as indacene, biphenylene, phenalene, anthracene, phenanthrene, and fluorene; tetracyclic aromatic hydrocarbons, such as fluoranthene, acephenanthrylene, aceanthrylene, triphenylene, pyrene, chrysene, tetraphene, tetracene, and pleiadene; and pentacyclic aromatic hydrocarbons, such as picene, perylene, pentaphene, pentacene, tetraphenylene, and naphthoanthracene. Preferable examples thereof include benzene, naphthalene, anthracene, phenanthrene, triphenylene, pyrene, chrysene, tetraphene, or tetracene. Examples of the non-substituted aromatic hydrocarbon group of L having 6 to 30 carbon atoms also include the same groups, but L is a divalent group.

Examples of the non-substituted π-electron excess heteroaromatic group having 4 to 30 carbon atoms include a heteroaromatic group having 4 to 30 carbon atoms and having a pyrrole ring, a thiophene ring, or a furan ring. Examples thereof include: nitrogen-containing aromatic groups having a pyrrole ring, such as pyrrole, pyrrolopyrrole, indole, pyrroloindole, benzoindole, naphthopyrrole, isoindole, pyrroloisoindole, benzoisoindole, naphthoisopyrrole, carbazole, benzocarbazole, indoloindole, carbazolocarbazole, and carbolise; sulfur-containing aromatic groups having a thiophene ring, such as thiophene, benzothiophene, naphthothiophene, dibenzothiophene, benzothienonaphthalene, benzothienobenzothiophene, benzothienodibenzothiophene, dinaphthothiophene, dinaphthothienothiophene, and naphthobenzothiophene; and oxygen-containing aromatic groups having a furan ring, such as furan, benzofuran, naphthofuran, dibenzofuran, benzofuronaphthalene, benzofurobenzofuran, benzofurodibenzofuran, dinaphthofuran, dinaphthofuranofuran, and naphthobenzofuran.

Preferable examples of the nitrogen-containing aromatic group having a pyrrole ring include carbazole, benzocarbazole, indoloindole, and carbazolocarbazole. Preferable examples of the sulfur-containing aromatic group having a thiophene ring include thiophene, dibenzothiophene, benzothienonaphthalene, benzothienobenzothiophene, benzothienodibenzothiophene, dinaphthothiophene, dinaphthothienothiophene, and naphthobenzothiophene. Preferable examples of the oxygen-containing aromatic group having a furan ring include dibenzofuran, benzofuronaphthalene, benzofurobenzofuran, benzofurodibenzofuran, dinaphthofuran, dinaphthofuranofuran, and naphthobenzofuran.

At least one of Ar¹ or Ar² preferably contains at least one substituted or non-substituted tricyclic fused skeleton. Examples of the tricyclic fused skeleton include azafluorene, azaphenanthrene, azaanthracene, carbazole, dibenzofuran, or dibenzothiophene. At least one of Ar¹ or Ar² preferably contains at least one or more carbazole, dibenzofuran, or dibenzothiophene skeletons, and more preferably contains at least one or more carbazole skeletons. These skeletons may have or may not have a substituent. Ar¹ or Ar² containing at least one substituted or non-substituted tricyclic fused skeleton is referred to, as a form where Ar¹ or Ar² represents a substituted or non-substituted π-electron excess heteroaromatic group having 4 to 30 carbon atoms or a substituted or non-substituted linked aromatic group formed by linking of two to six aromatic rings of aromatic groups selected from a substituted or non-substituted aromatic hydrocarbon group having 6 to 30 carbon atoms and the π-electron excess heteroaromatic group, a case where Ar¹ or Ar² contains at least one of these skeletons.

The group can be a π-electron excess heteroaromatic group in which rings of two or more kinds of groups selected from the nitrogen-containing aromatic group, the sulfur-containing aromatic ring, the oxygen-containing aromatic group, and the like are fused. Examples of such a π-electron excess heteroaromatic group include: groups in which an aromatic group having a pyrrole ring and an aromatic group having a furan ring are fused, such as benzofurocarbazole and benzofurobenzocarbazole; groups in which an aromatic group having a pyrrole ring and an aromatic group having a thiophene ring are fused, such as benzothienocarbazole and benzothienobenzocarbazole; and groups in which an aromatic group having a furan ring and an aromatic group having a thiophene ring are fused, such as benzofurodibenzothiophene and benzofurobenzocarbazole. Examples of the non-substituted π-electron excess heteroaromatic group of L include the same groups, but L is a divalent group.

Ar¹, Ar², or L can be a linked aromatic group generated by linking two to six of the aromatic hydrocarbon groups or the π-electron excess heteroaromatic groups.

The linked aromatic group herein is referred to an aromatic group in which carbons in aromatic rings of aromatic groups (referred to an aromatic hydrocarbon group or a π-electron excess heteroaromatic group) are linked with each other with a single bond. The linking structure may be linear or branched. The aromatic group may be the aromatic hydrocarbon group or the π-electron excess heteroaromatic group, and a plurality of the aromatic groups may be same as or different from each other. The aromatic group corresponding to the linked aromatic group differs from the substituted aromatic group.

Examples of a substituent that the aromatic hydrocarbon group, the π-electron excess heteroaromatic group, and the linked aromatic group can have include an alkyl group having 1 to 20 carbon atoms. The alkyl group having 1 to 20 carbon atoms may be any of linear, branched, and cyclic alkyl groups, and is preferably a linear, branched, or cyclic alkyl group having 1 to 10 carbon atoms.

Specific examples of the substituent include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, a 4-butylcyclohexyl group, and a 4-dodecylcyclohexyl group.

Preferable specific examples of the material for a photoelectric conversion element of the present invention represented by the general formula (1) are shown below, but the material is not limited thereto.

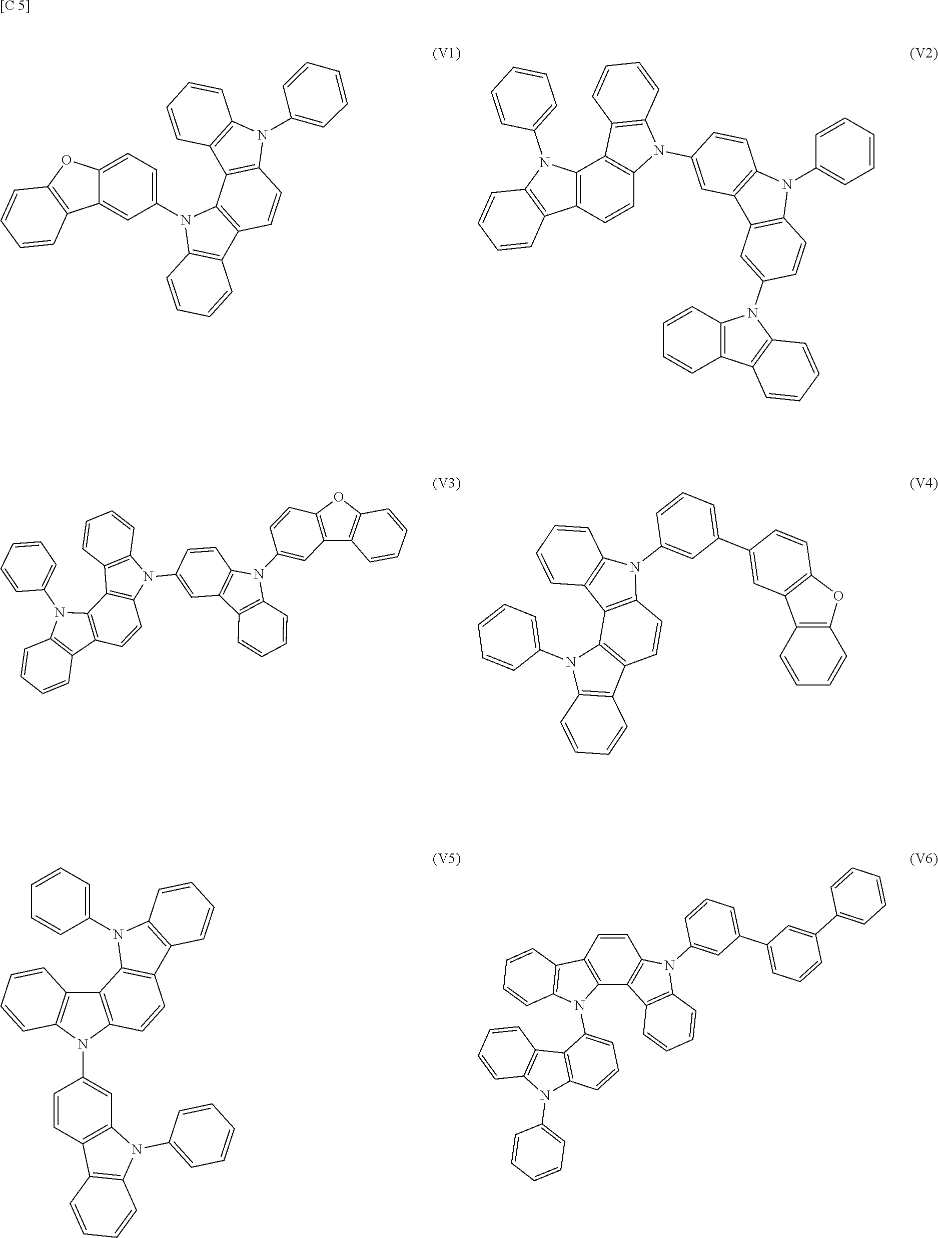

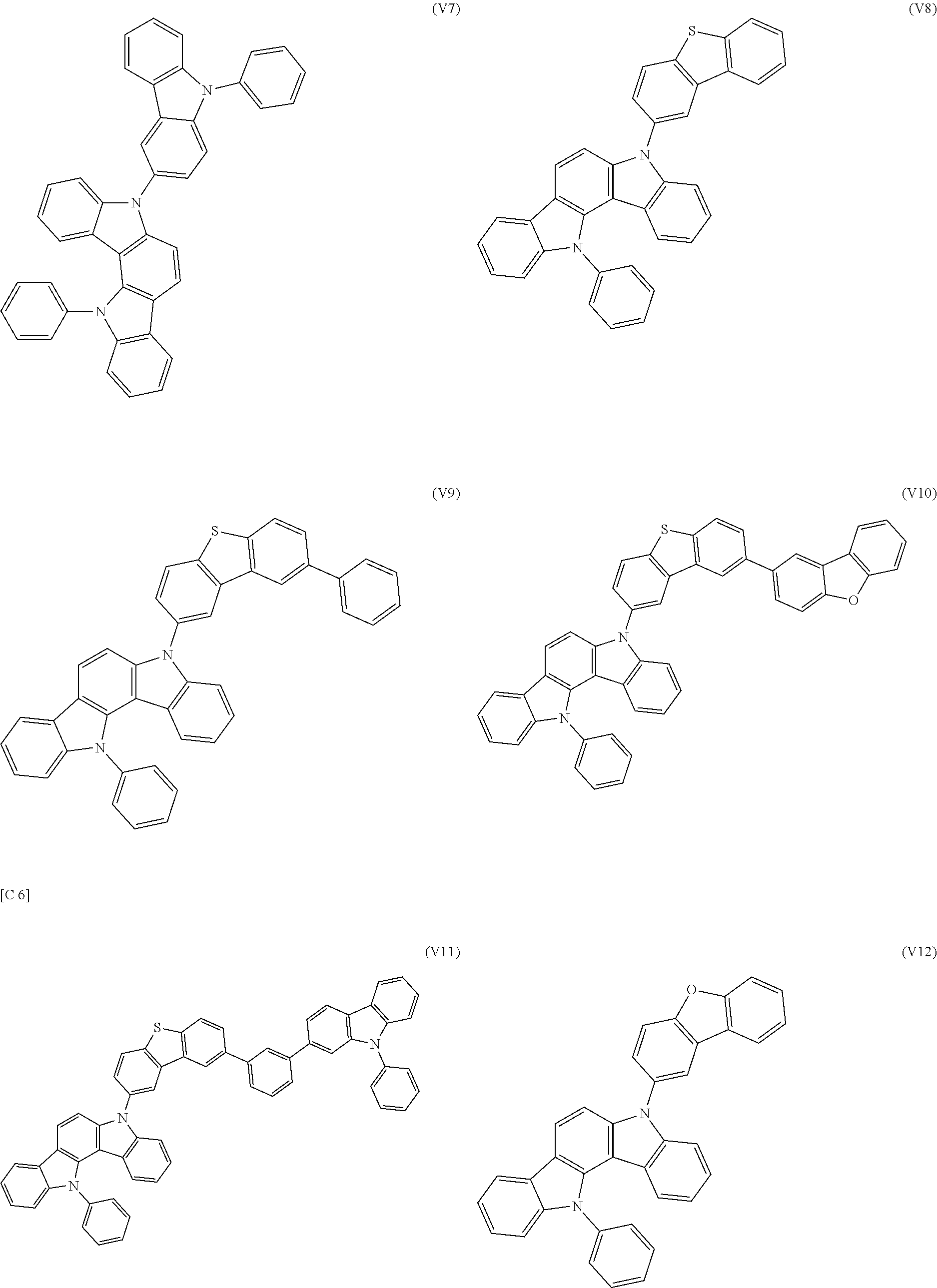

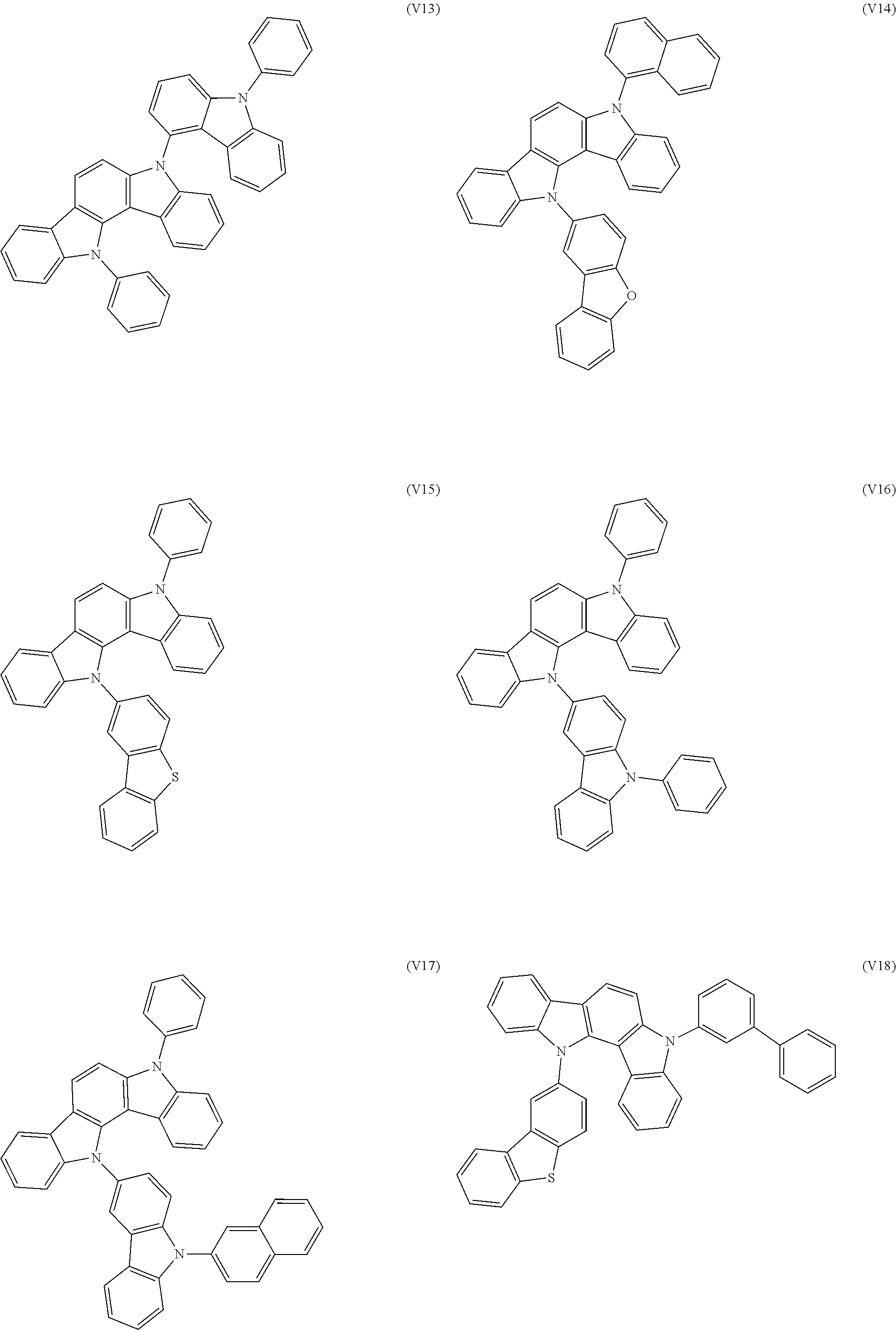

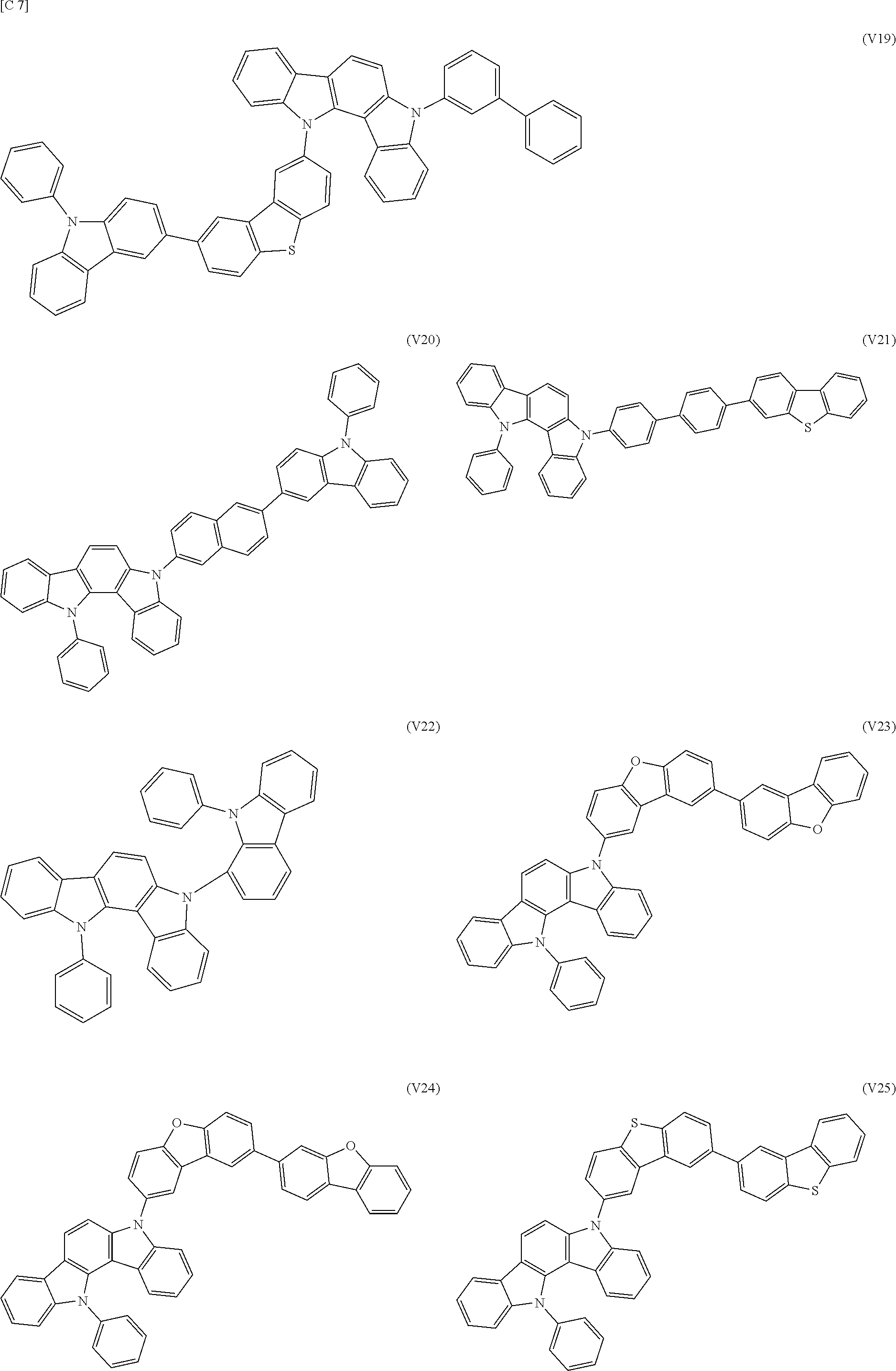

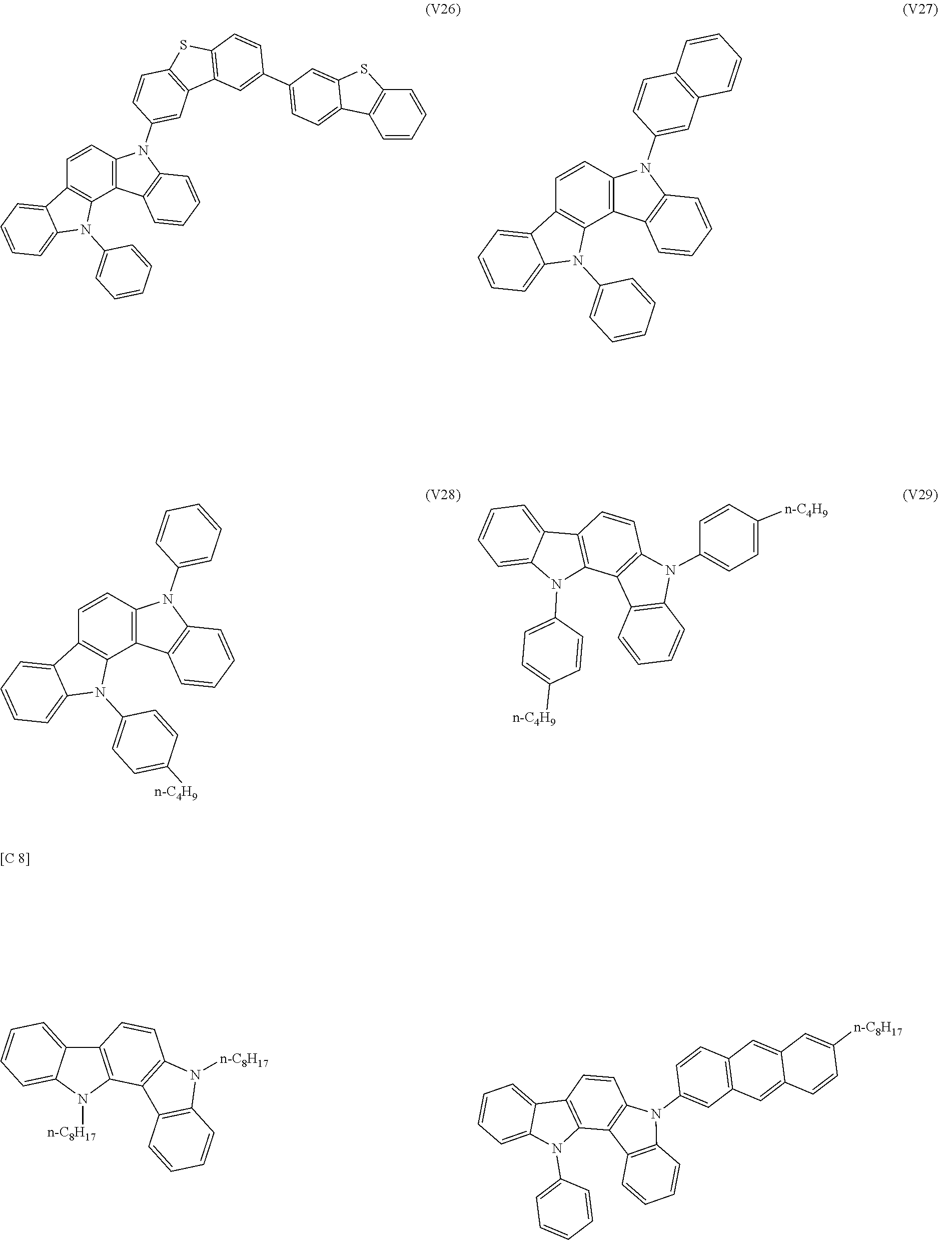

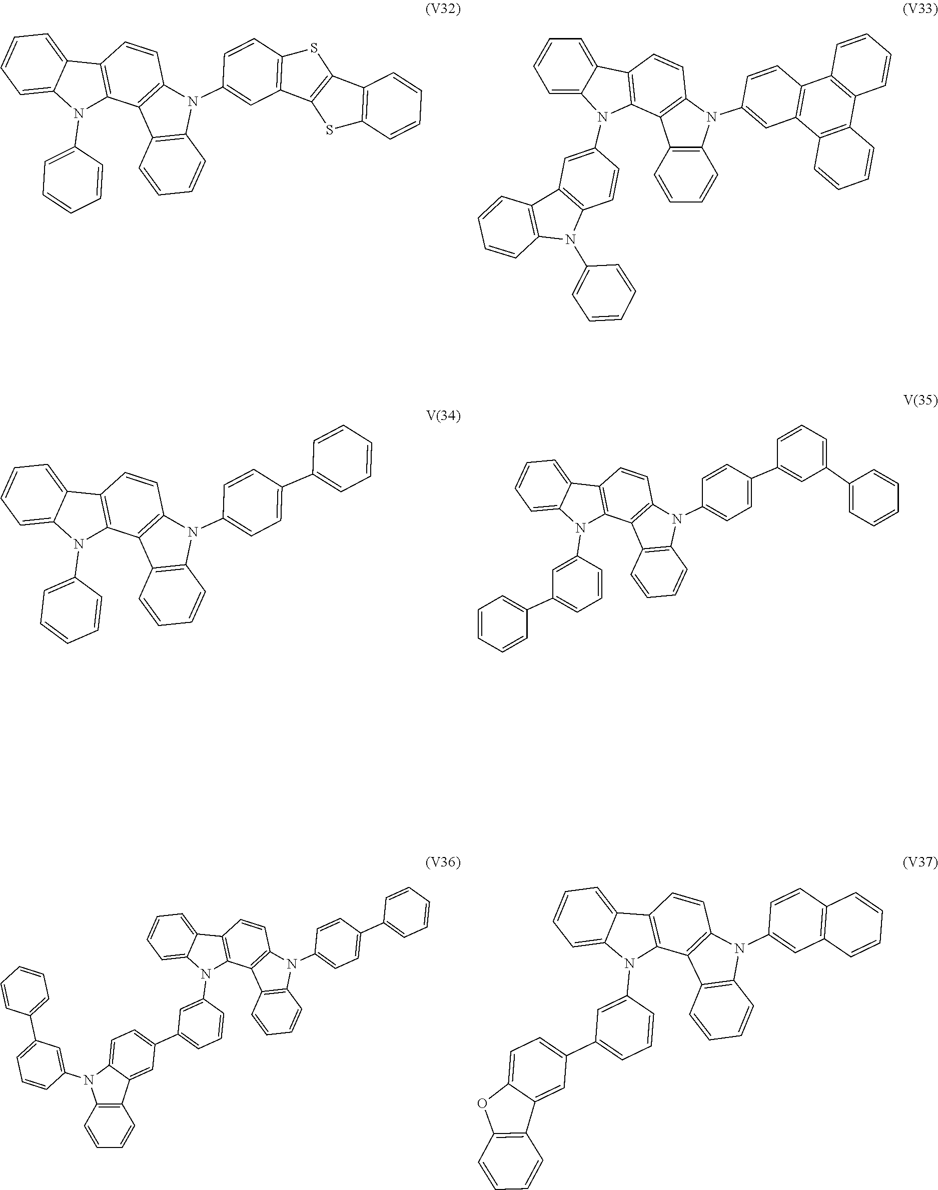

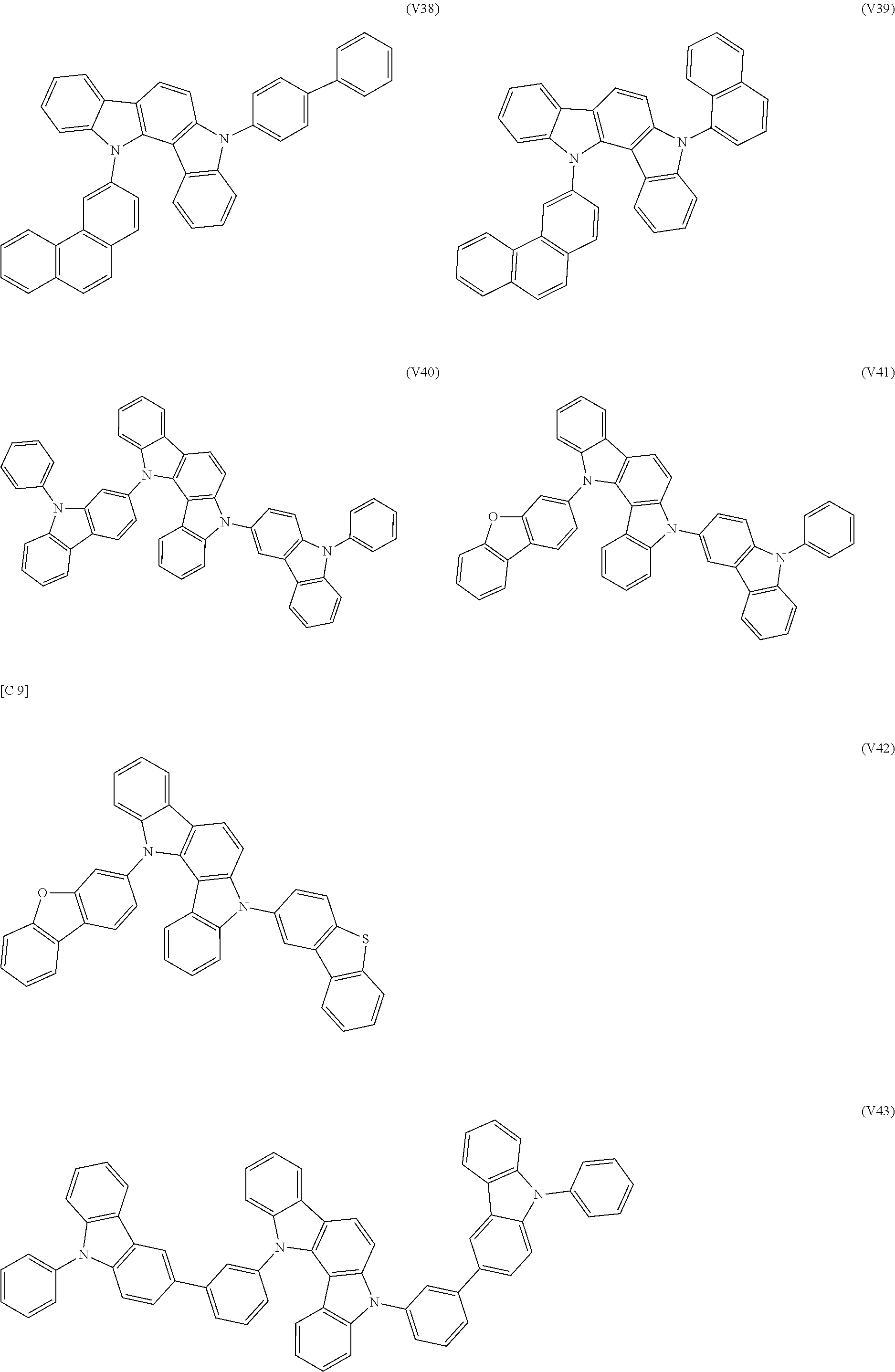

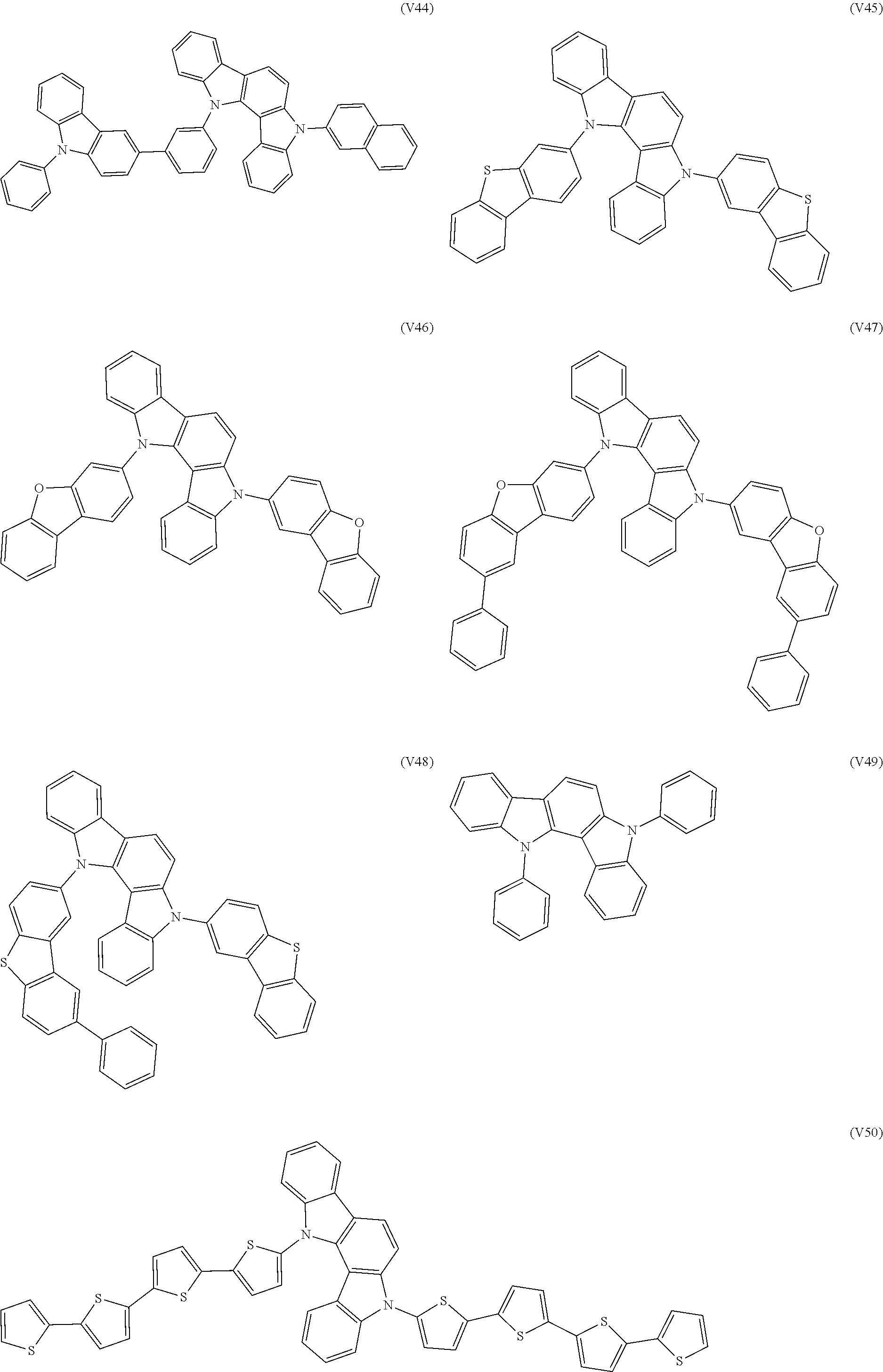

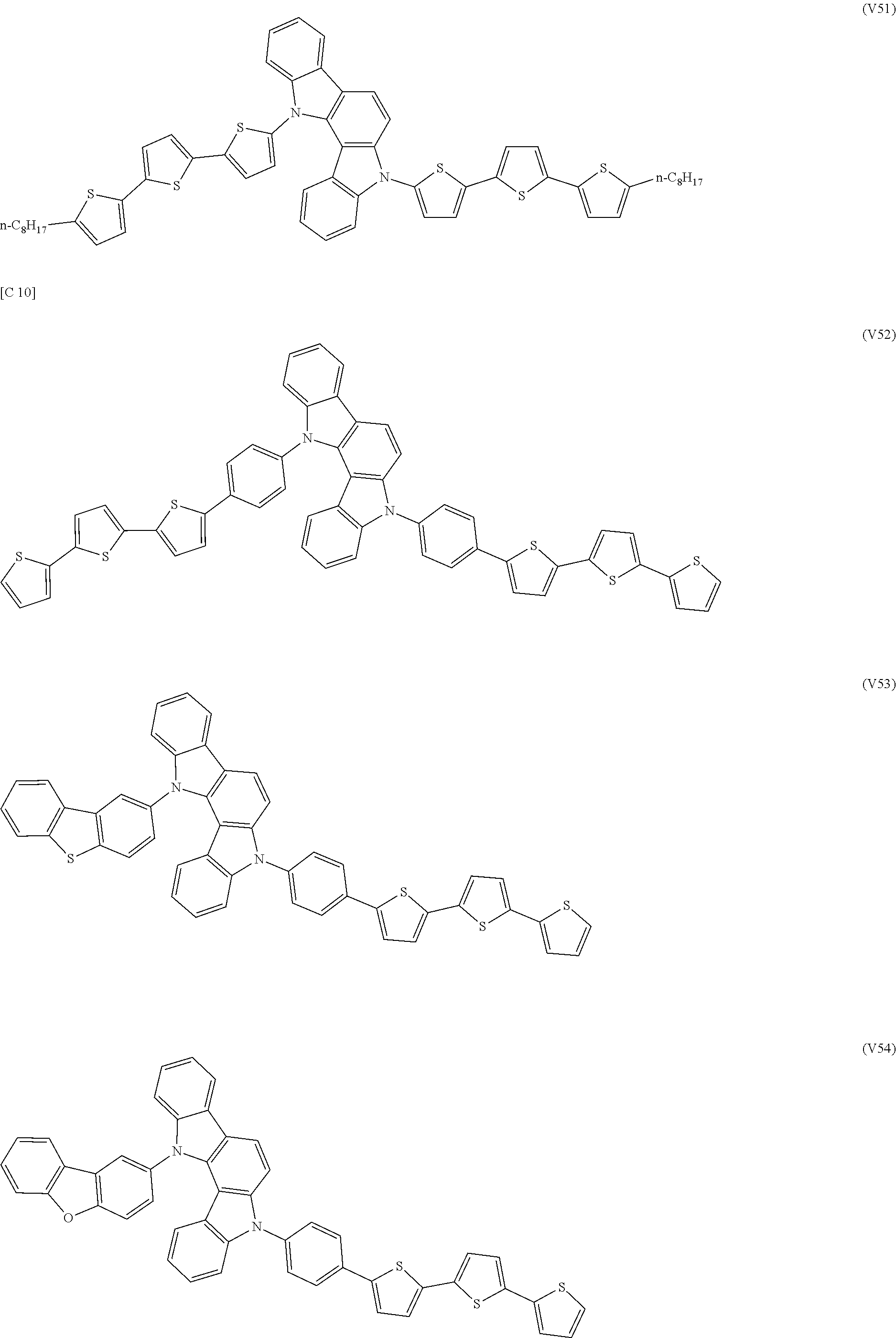

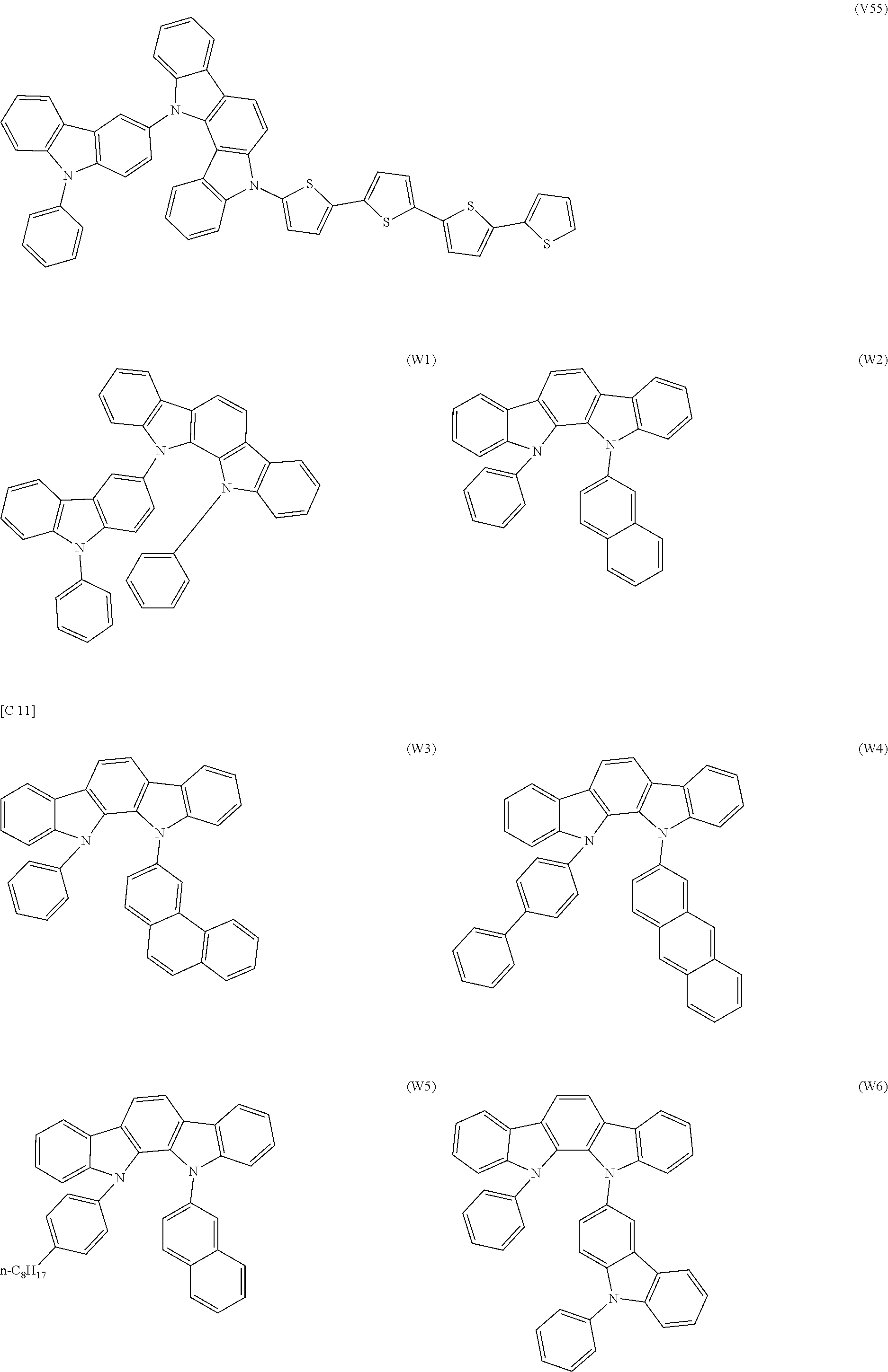

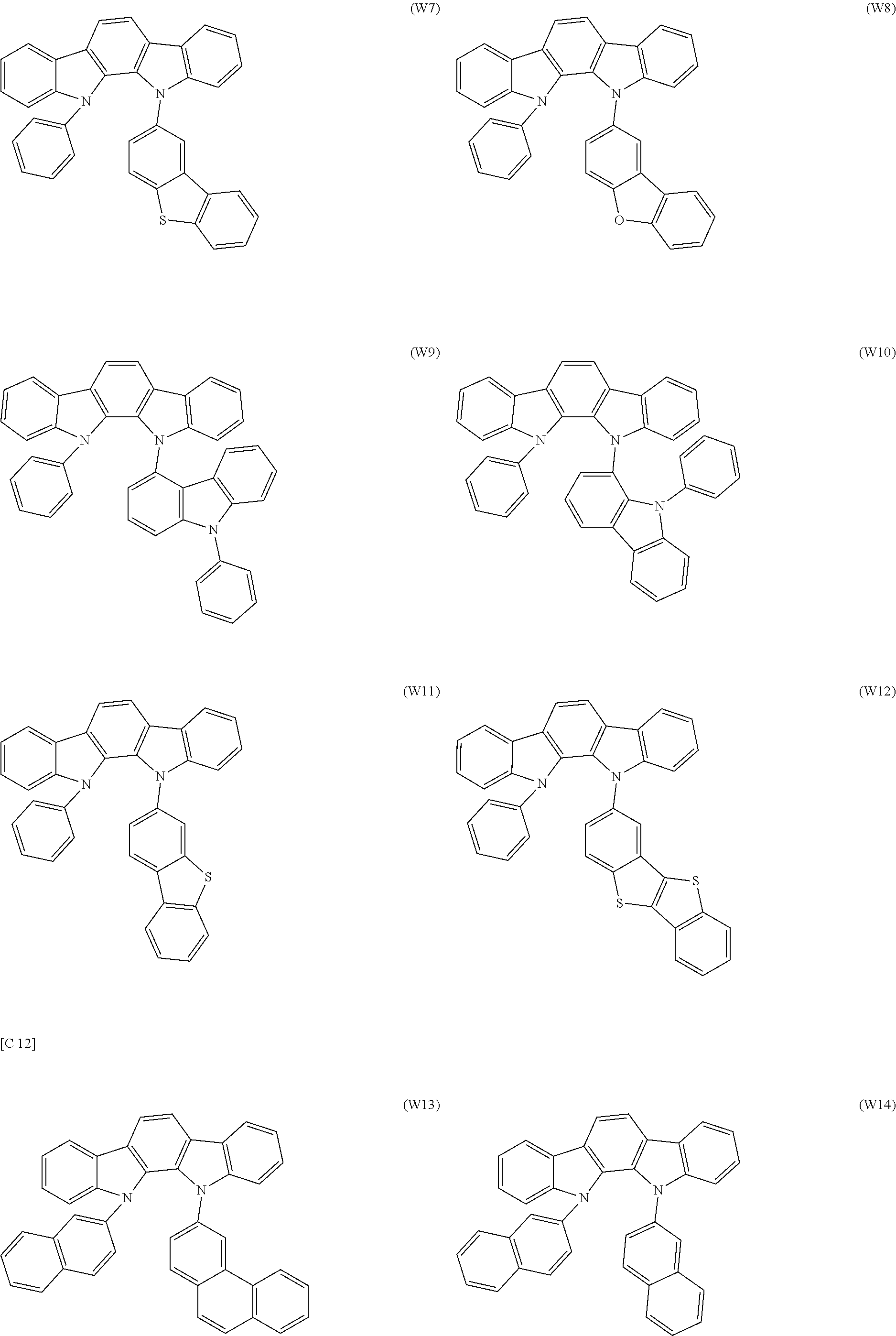

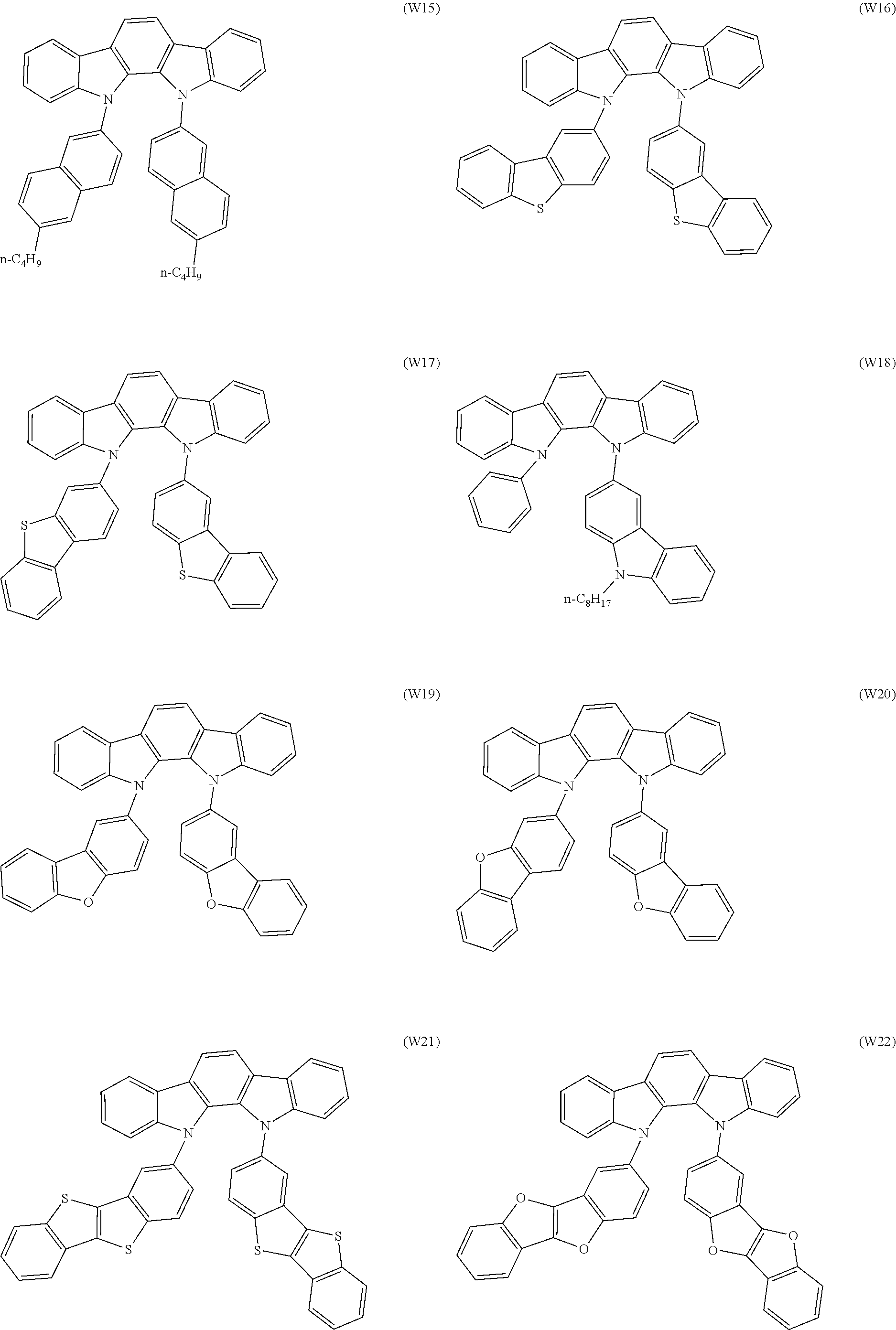

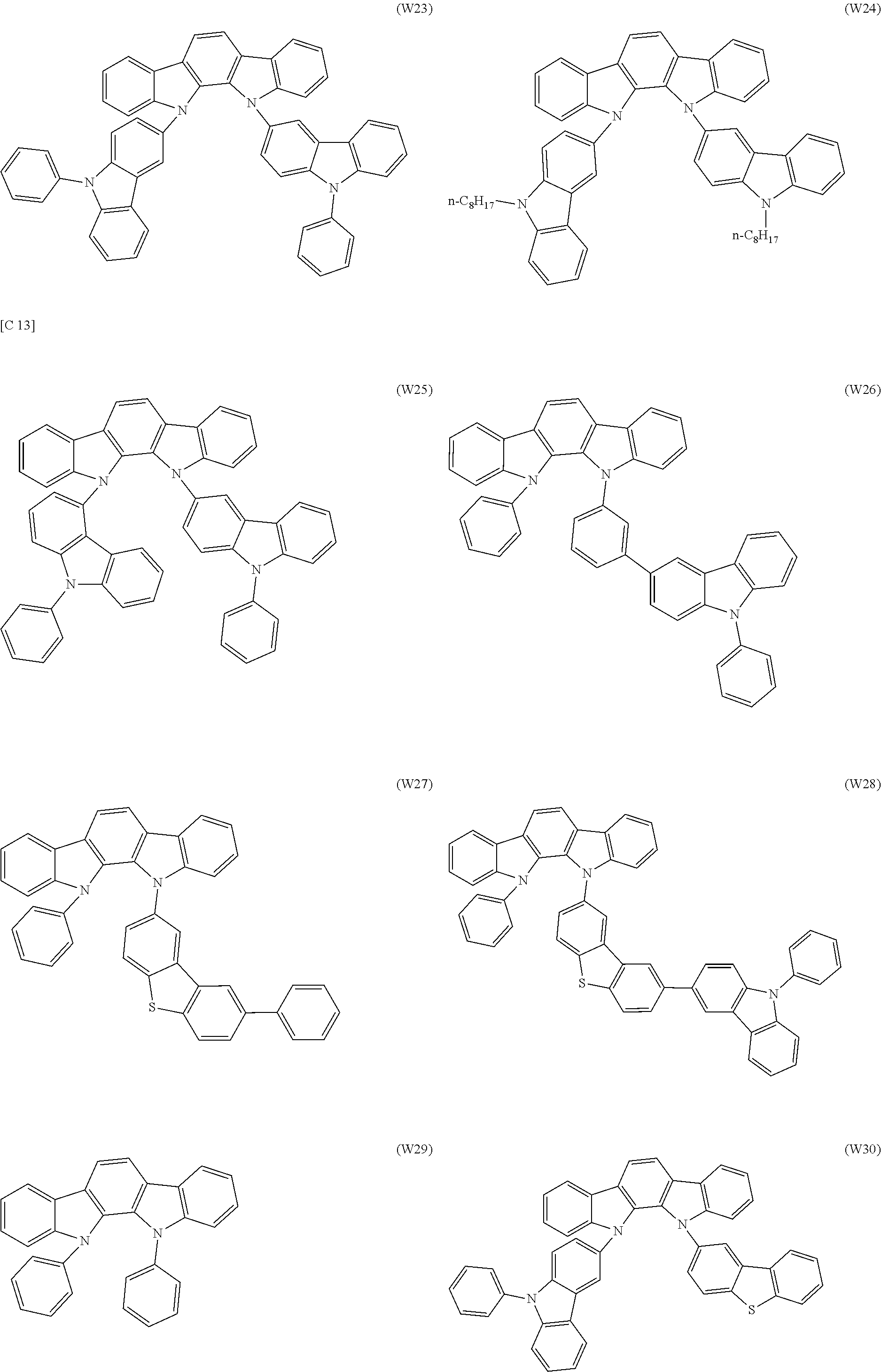

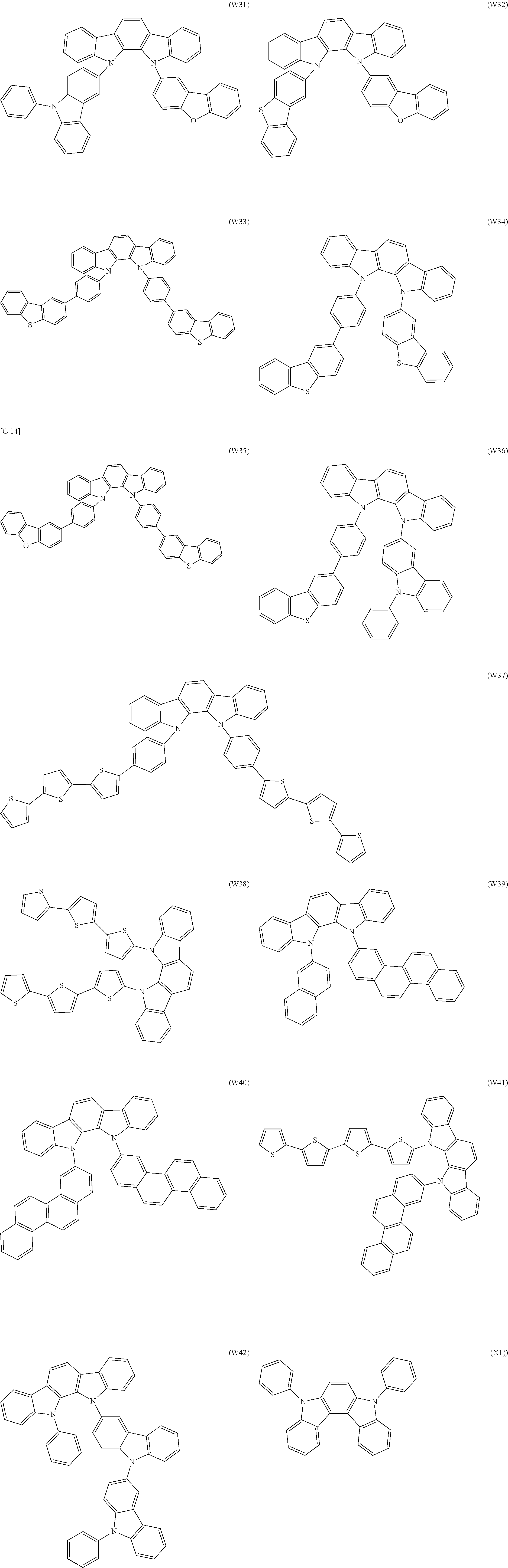

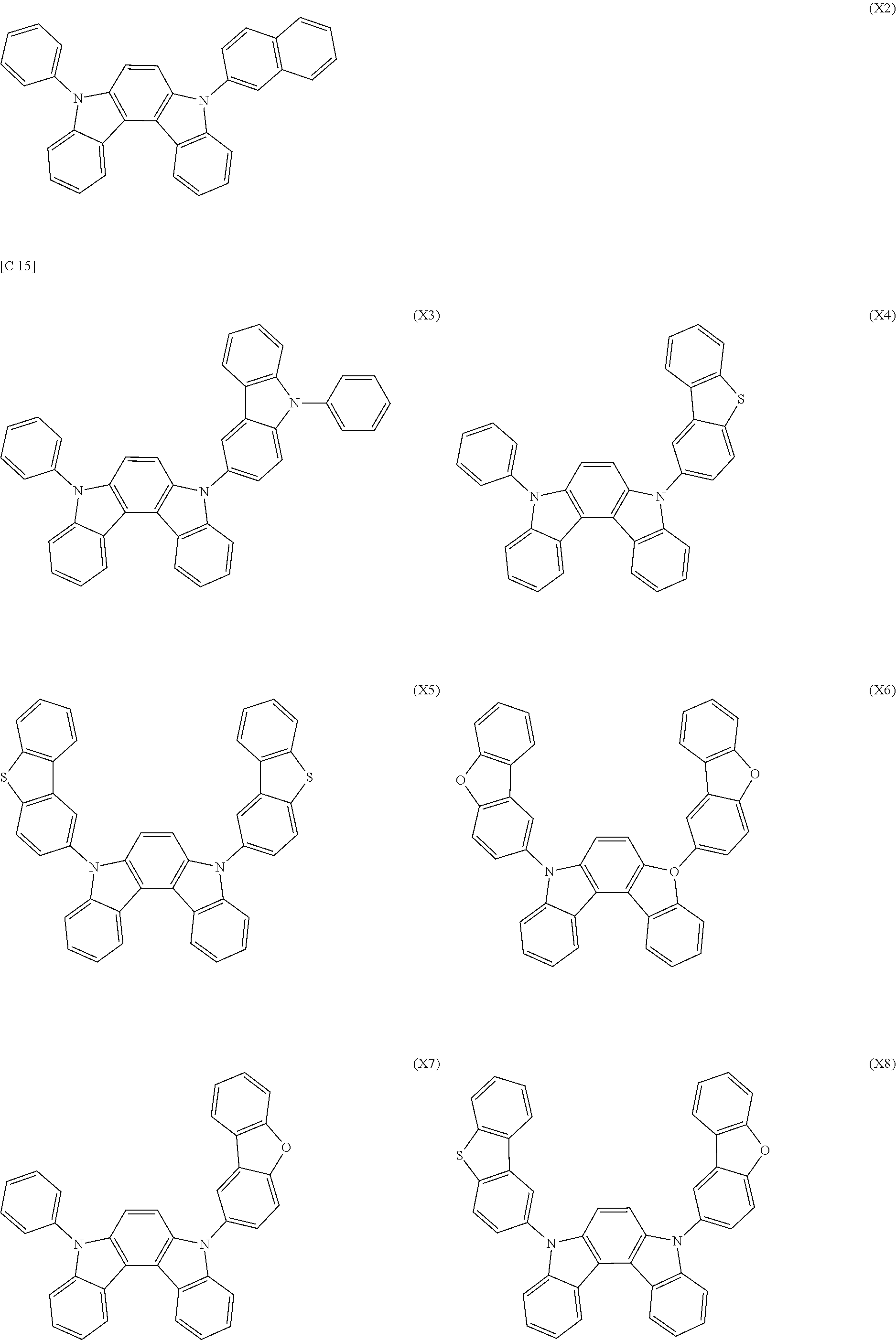

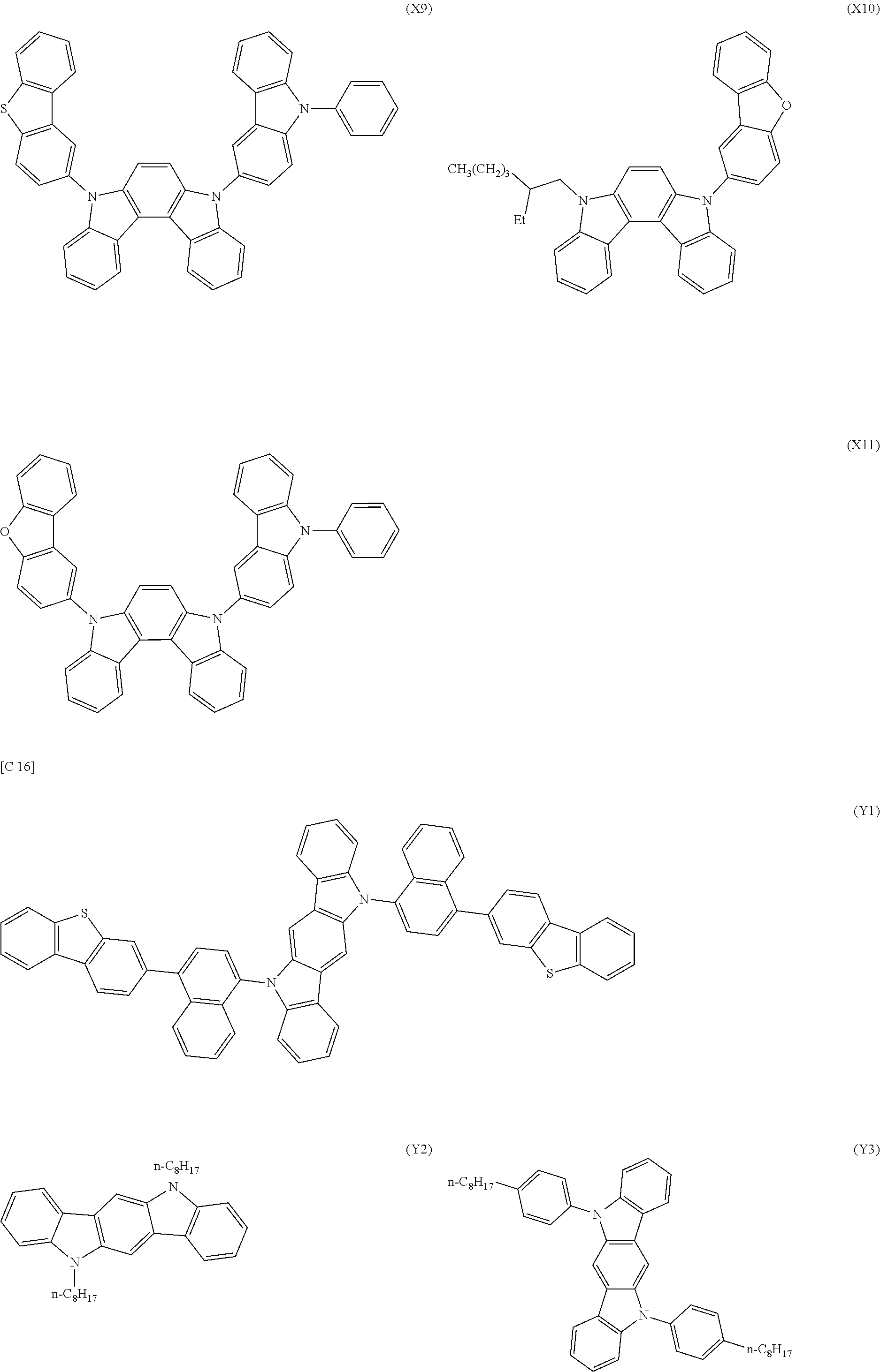

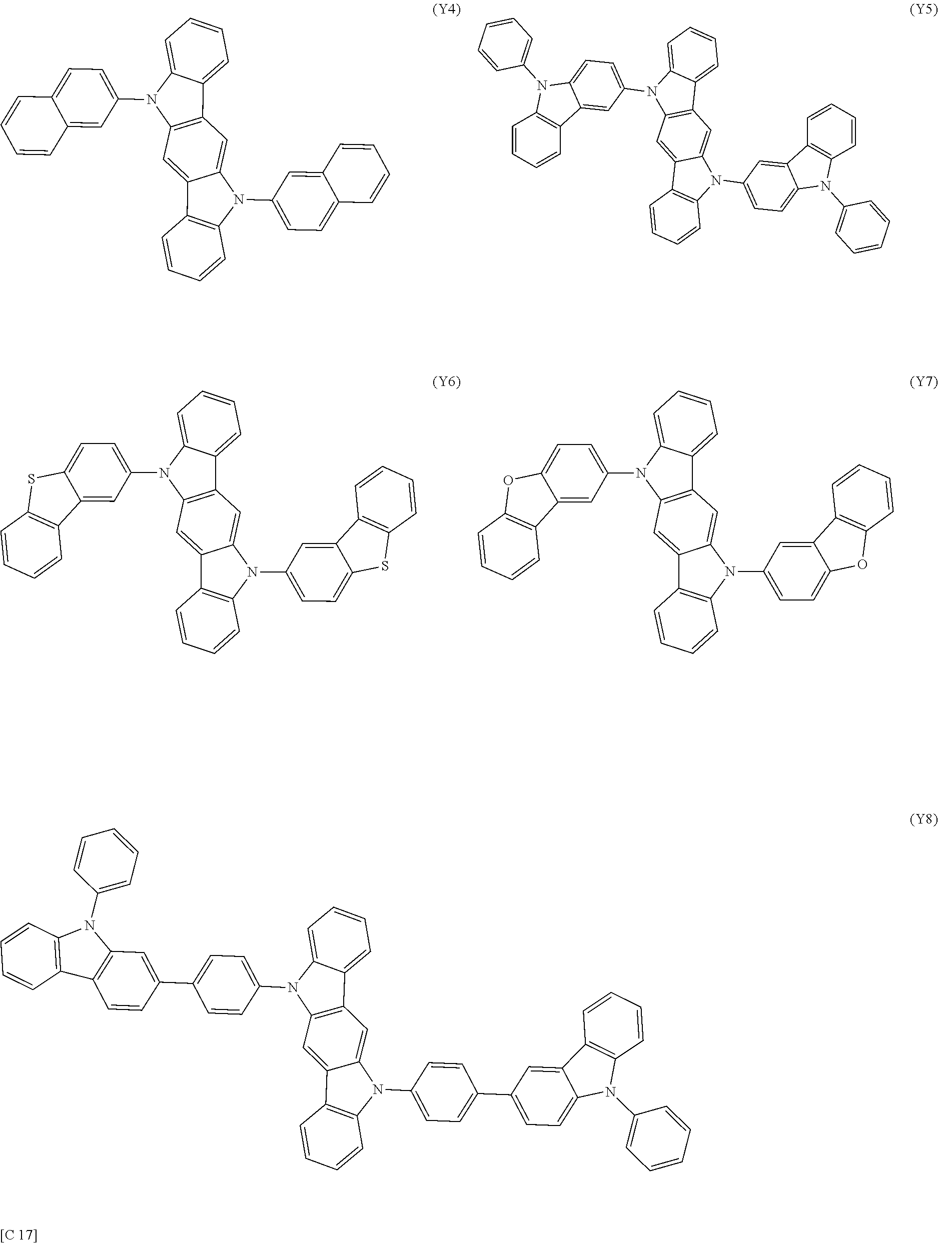

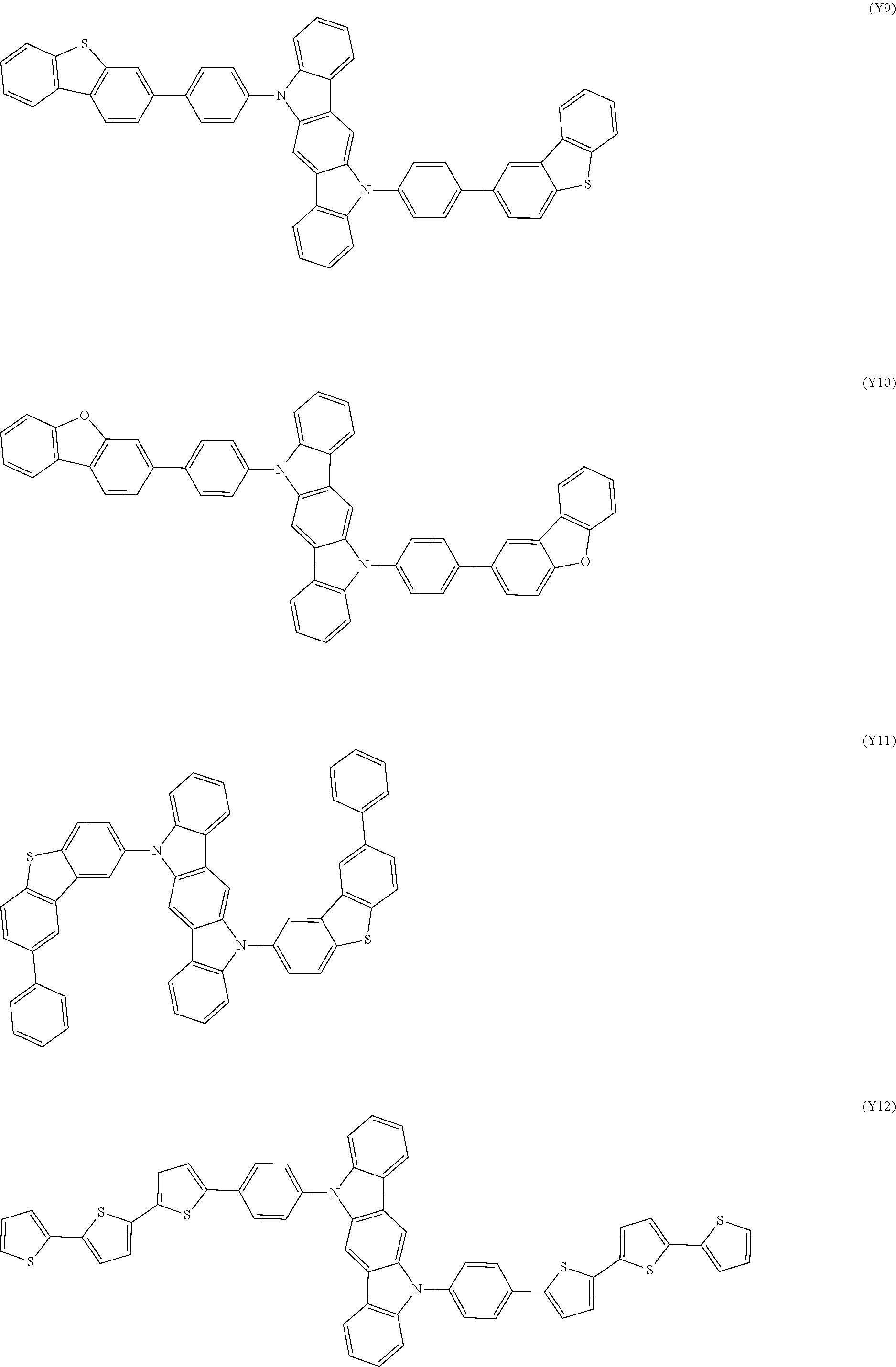

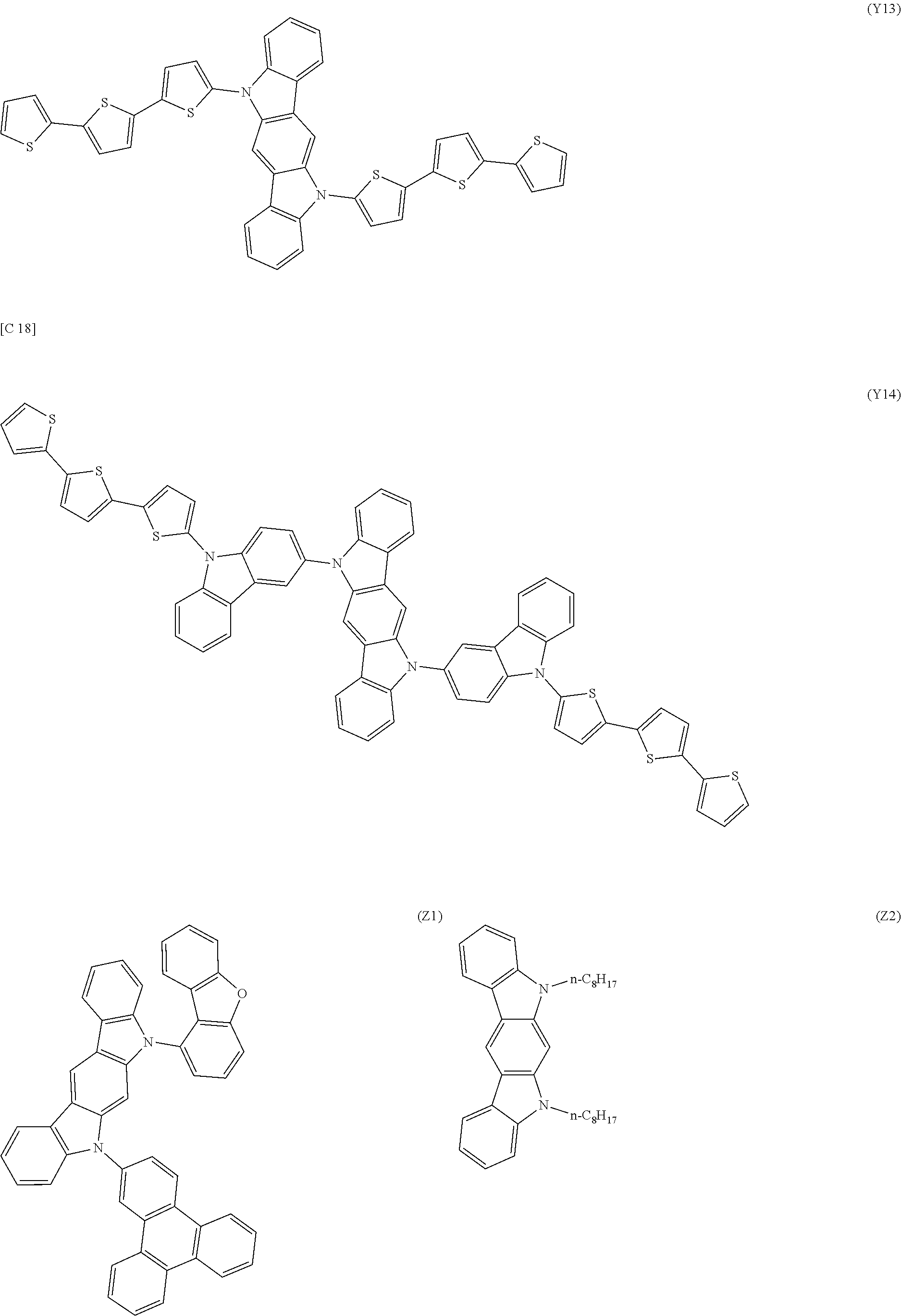

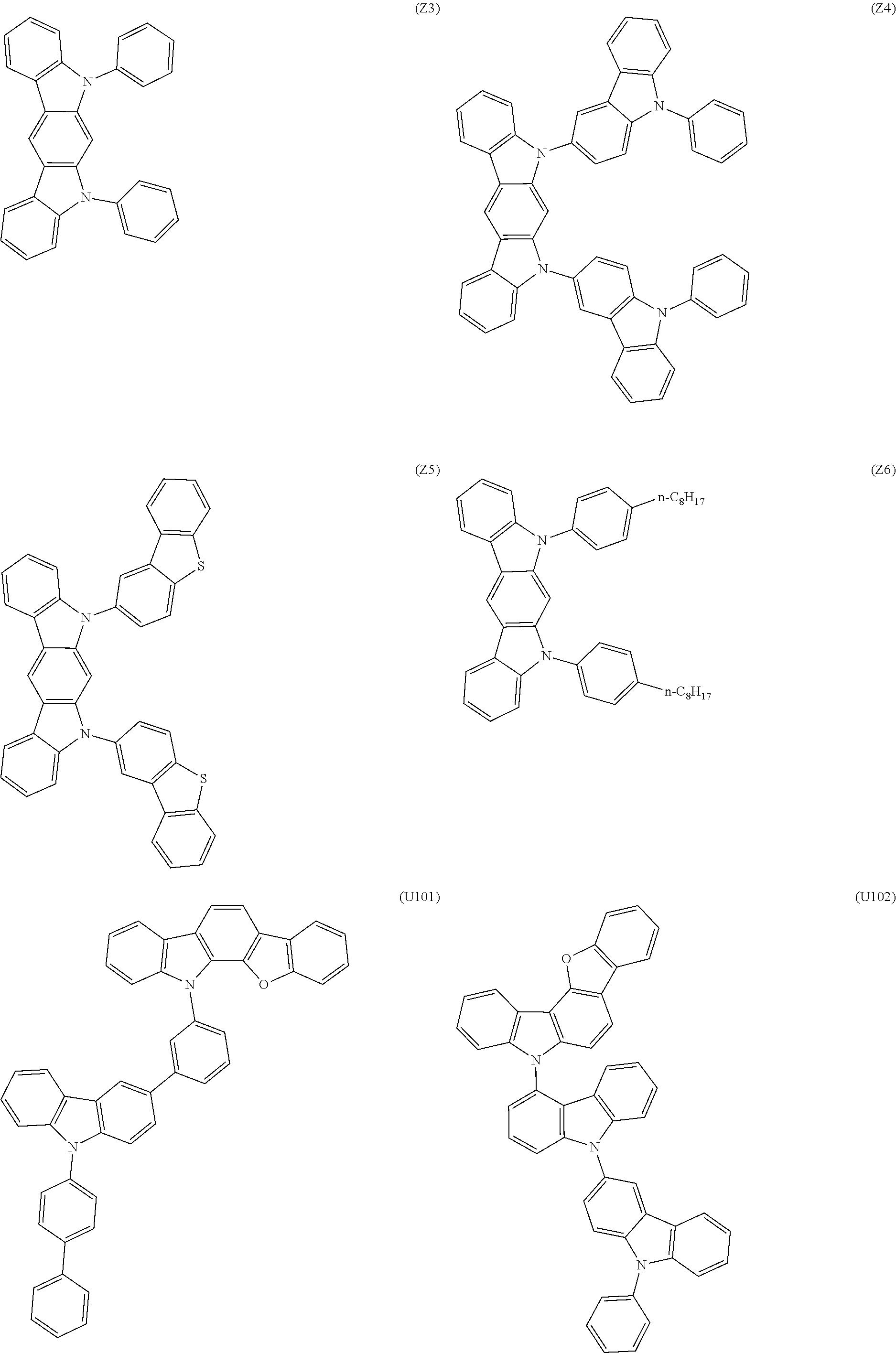

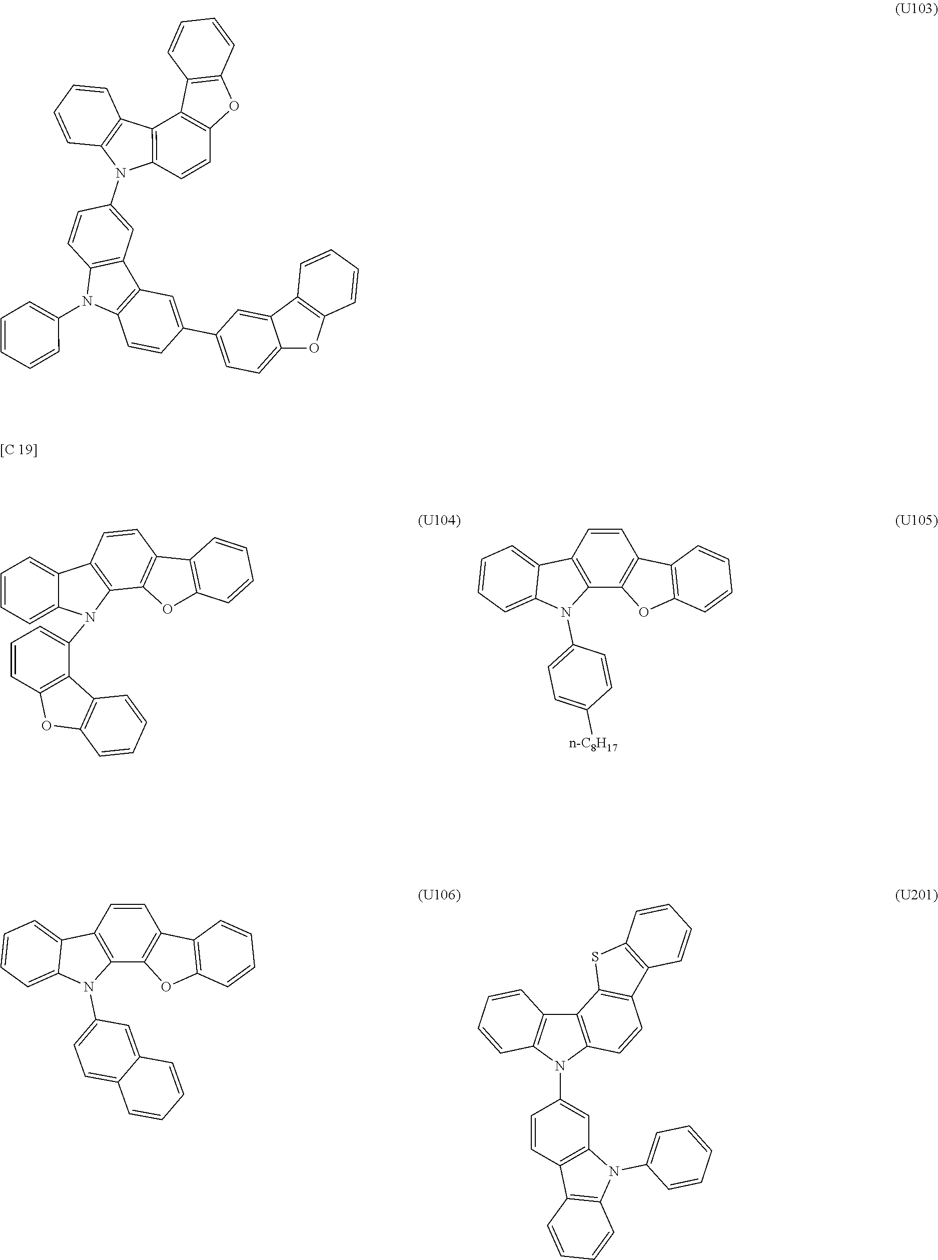

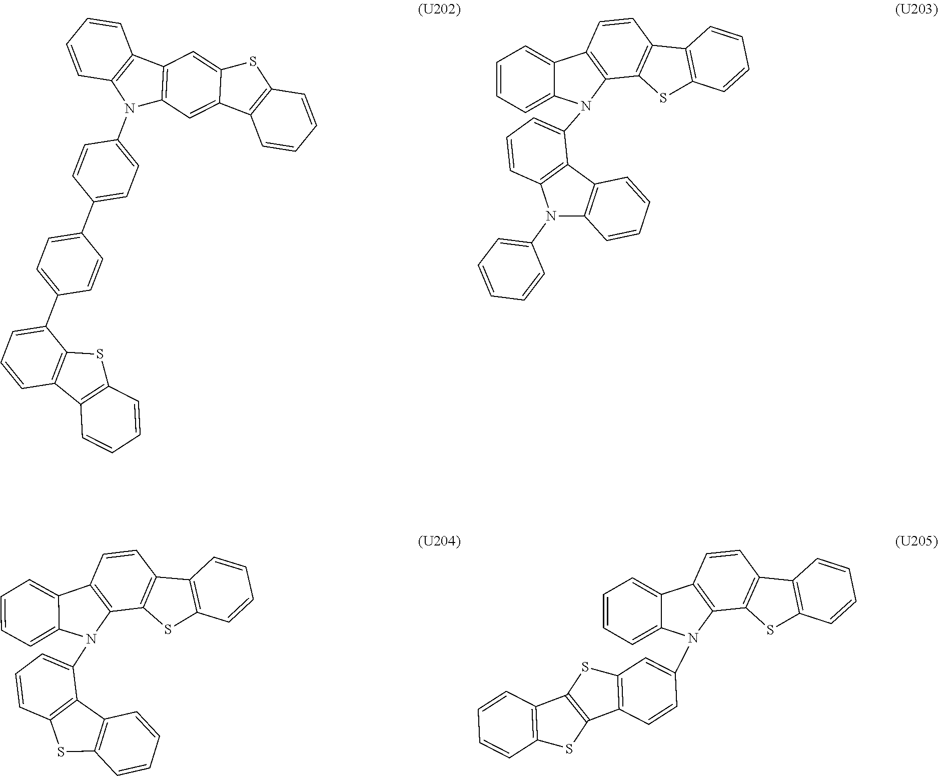

Preferable specific examples of the material for a photoelectric conversion element of the present invention represented by the general formula (2) are shown below, but the material is not limited thereto.

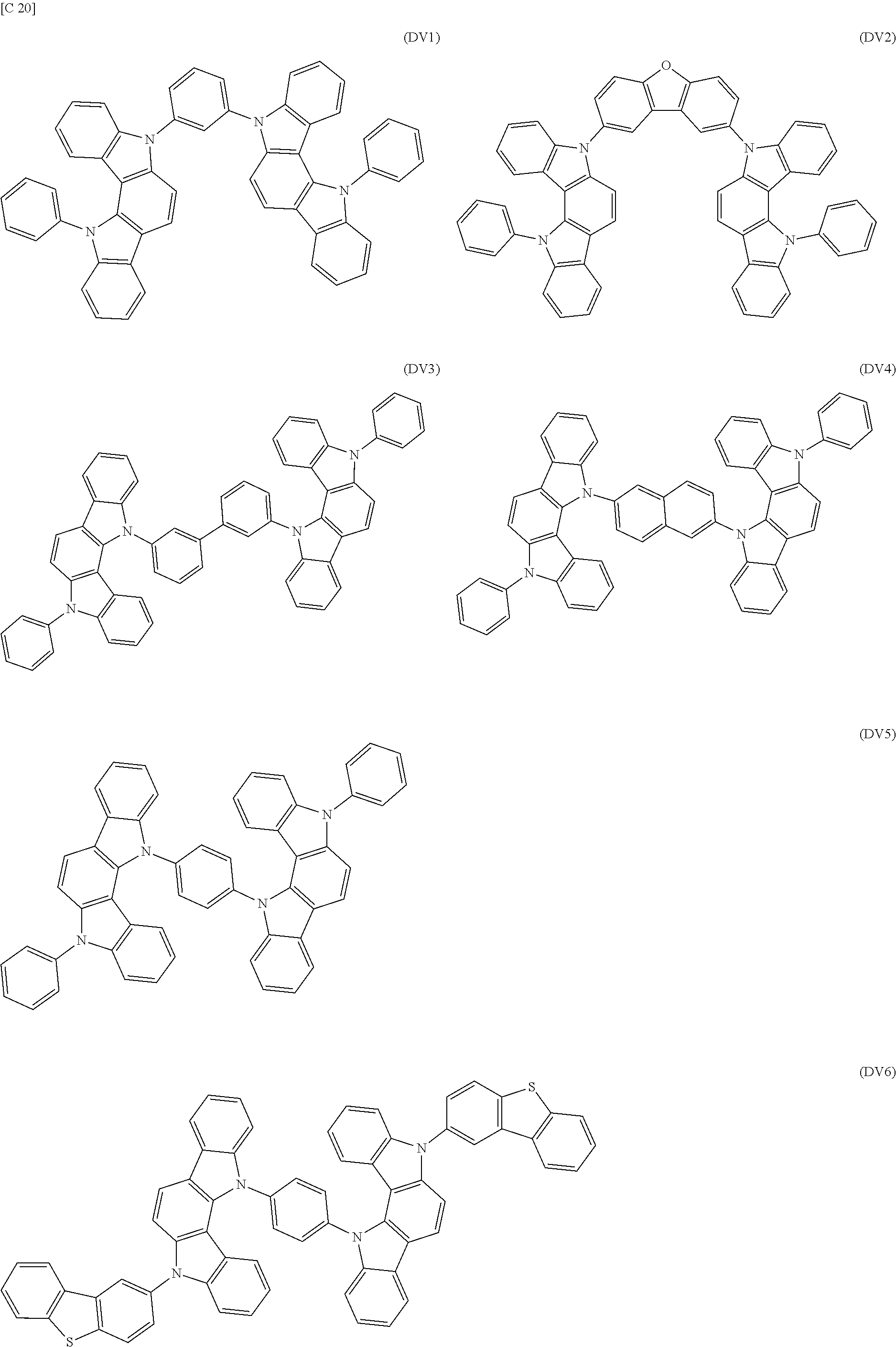

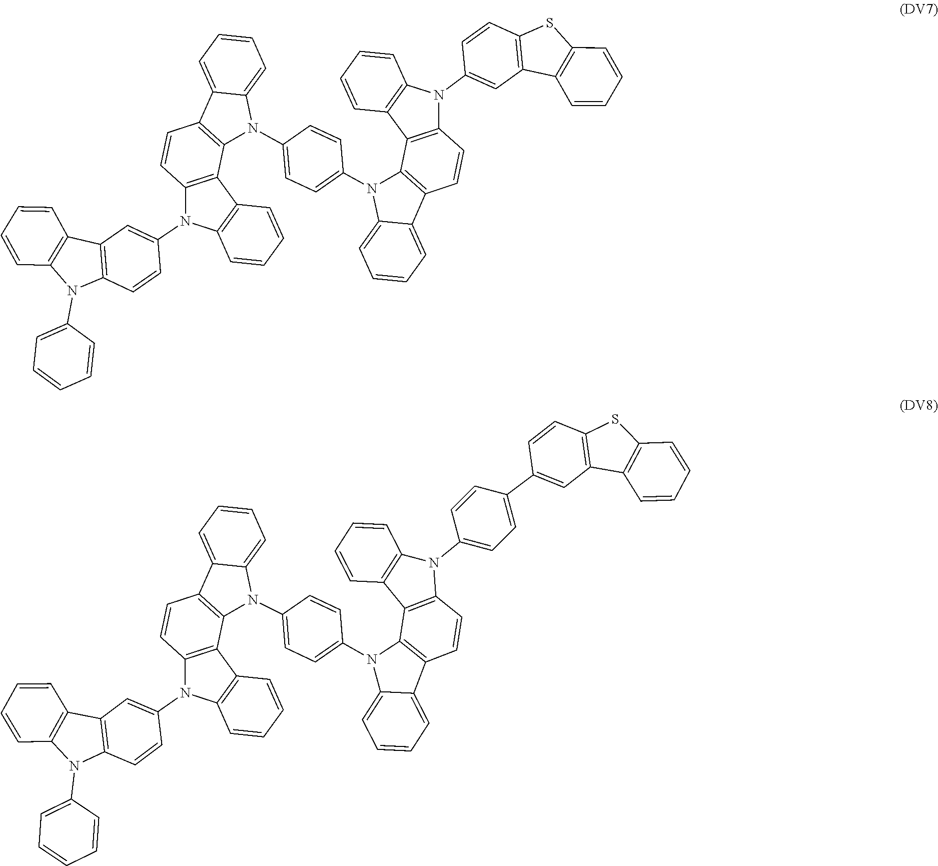

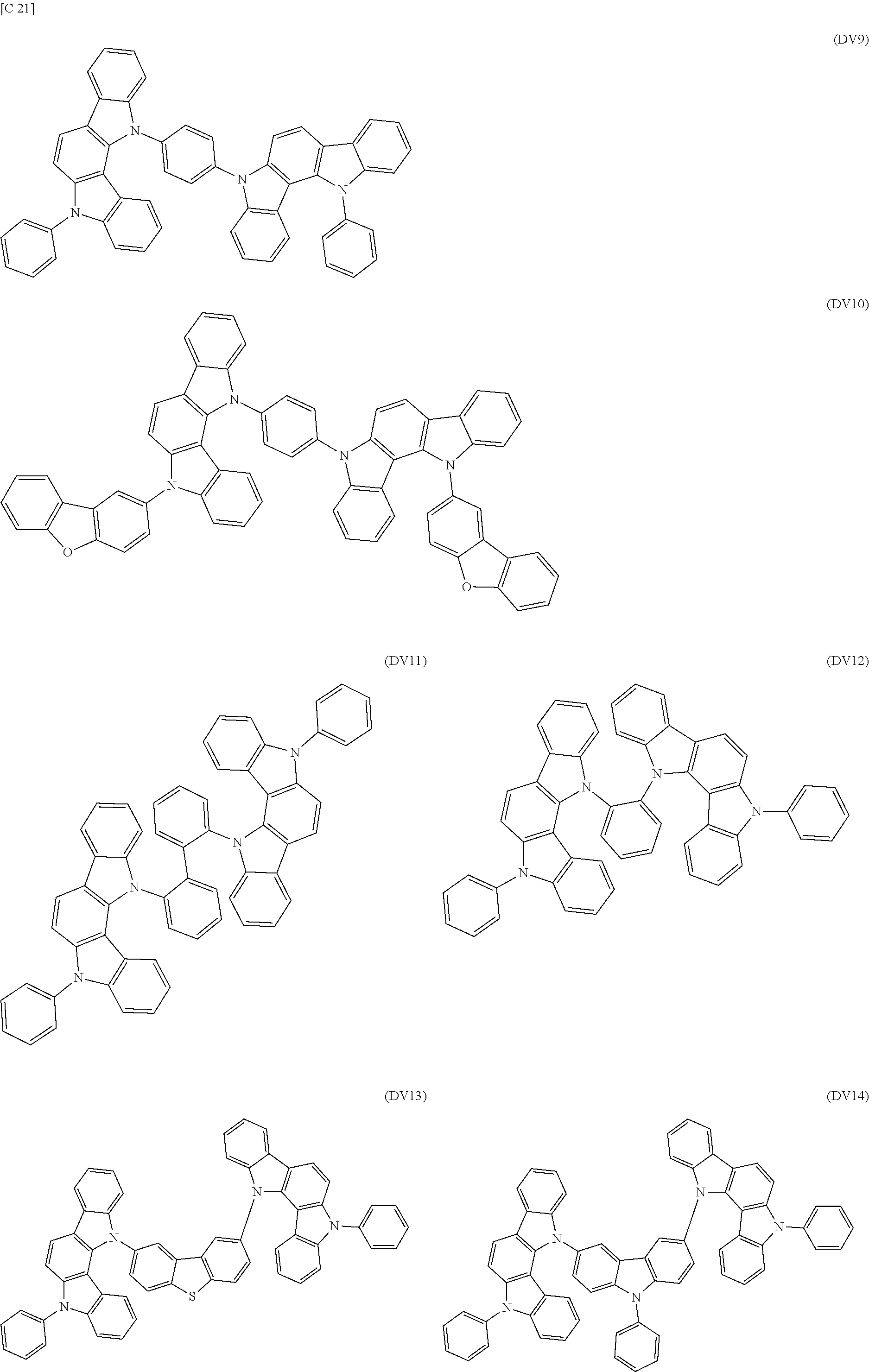

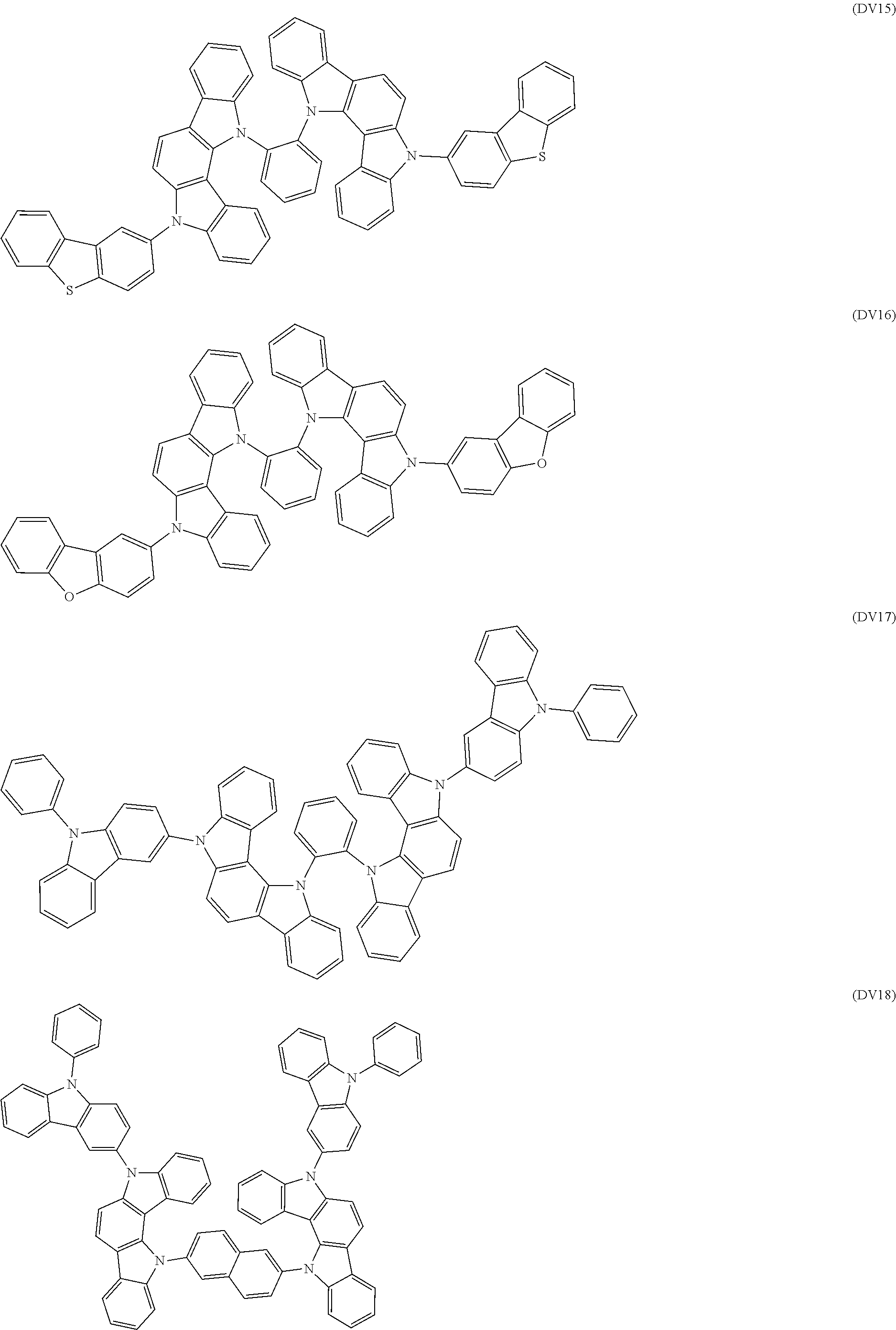

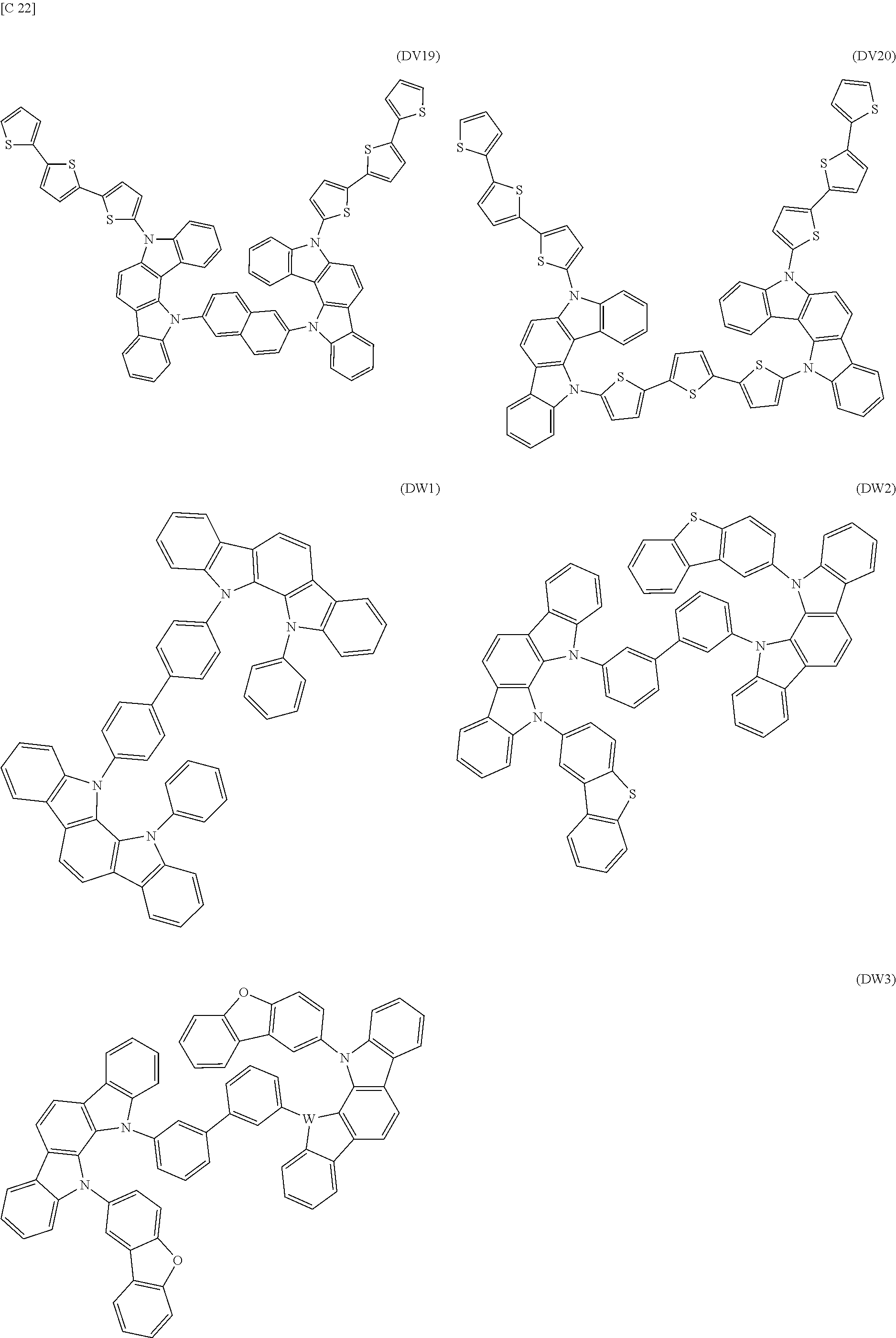

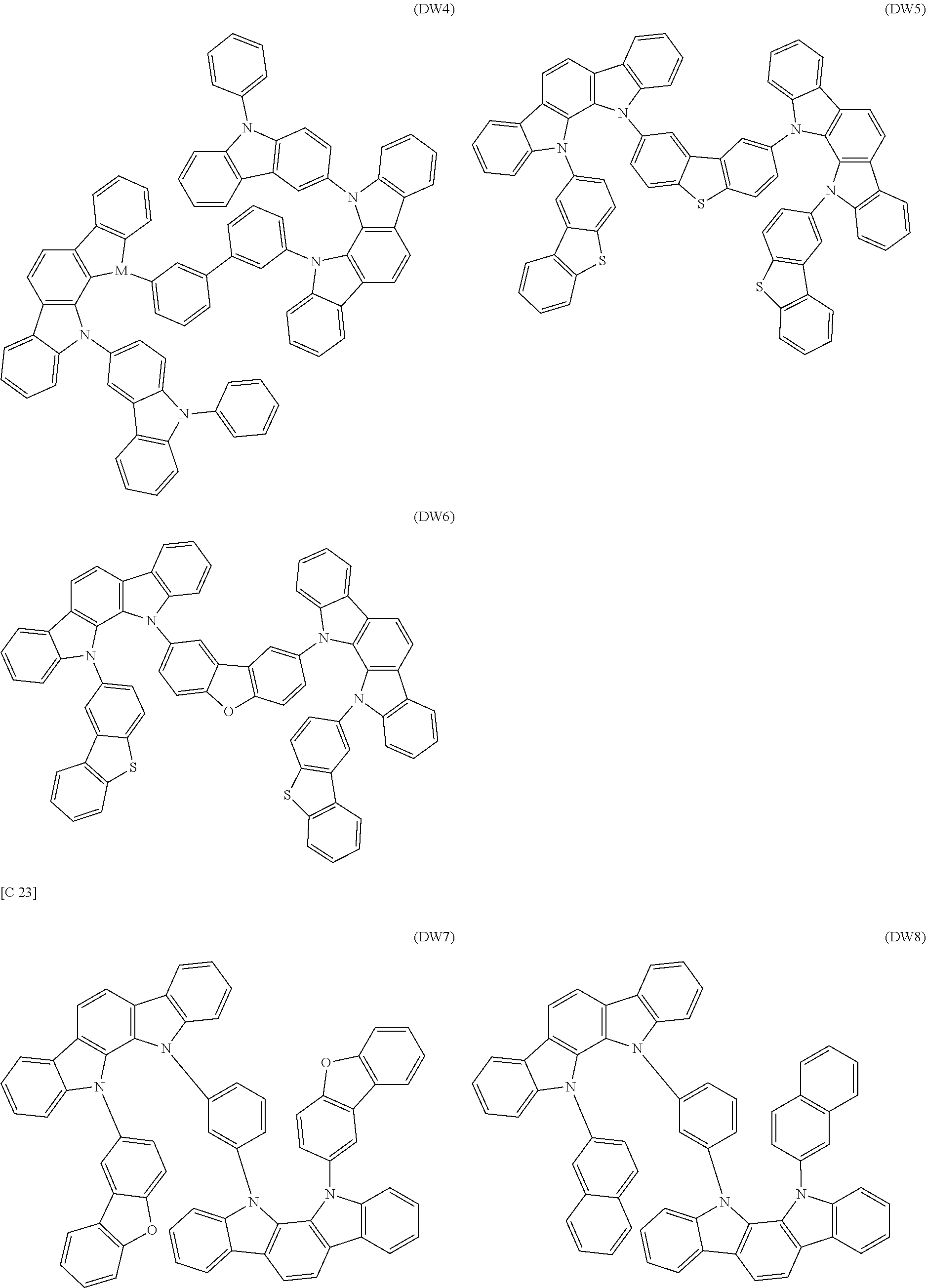

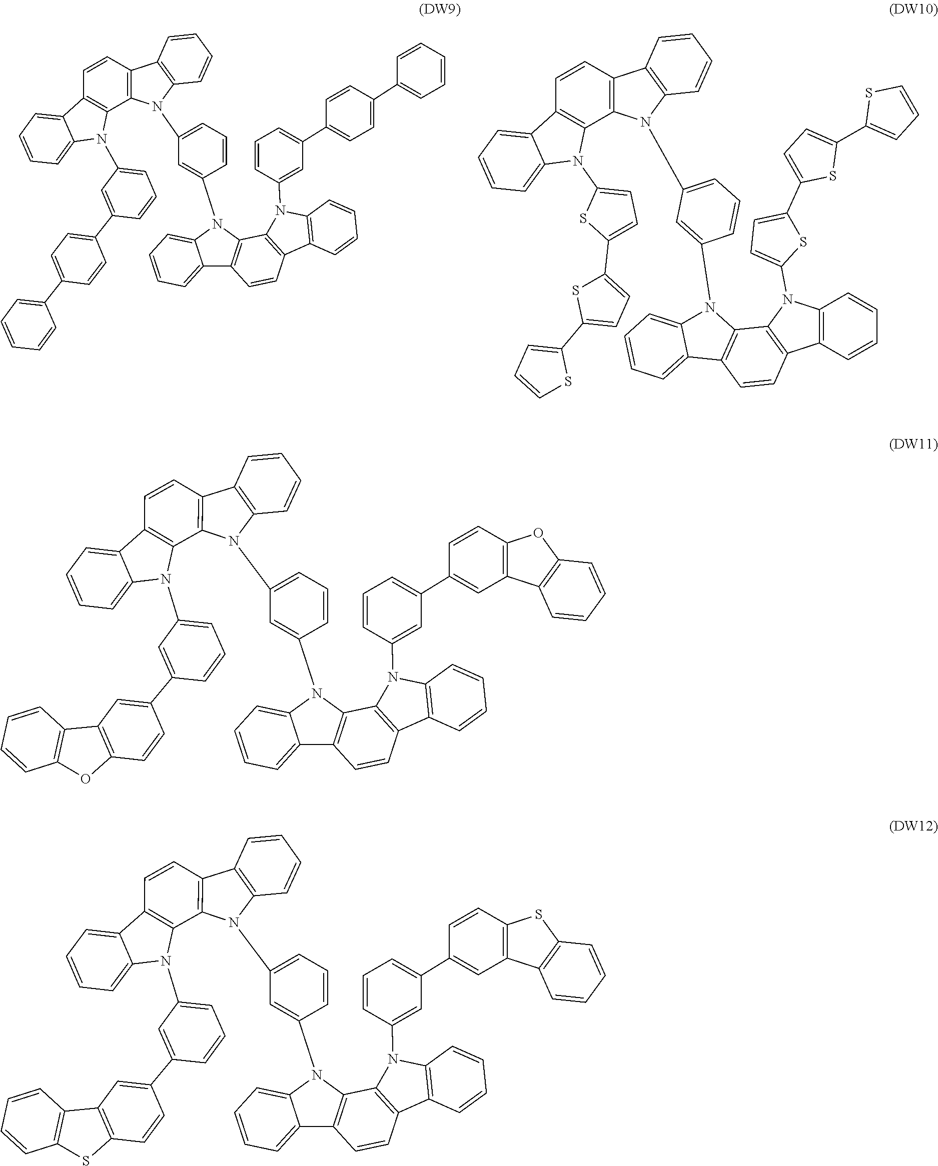

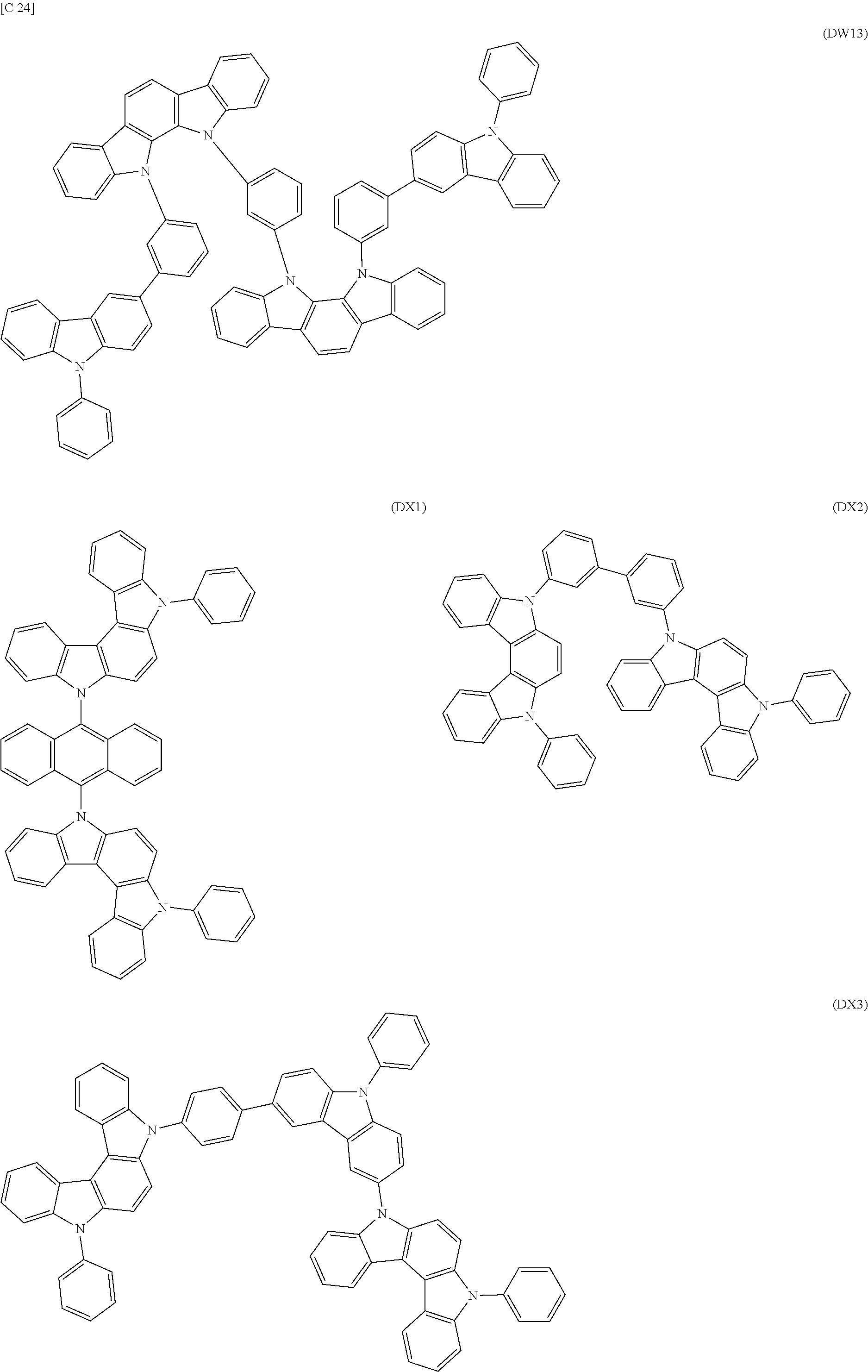

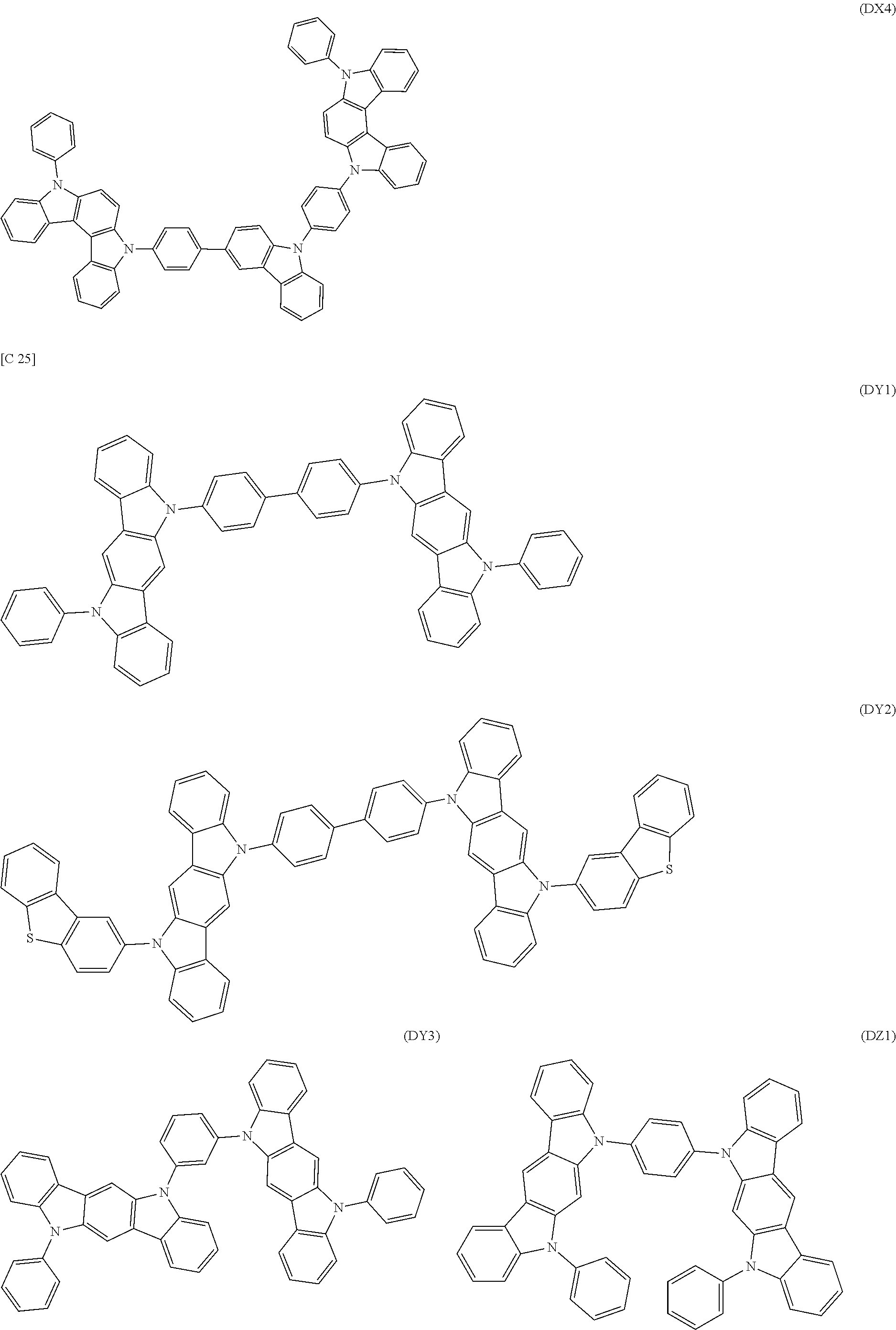

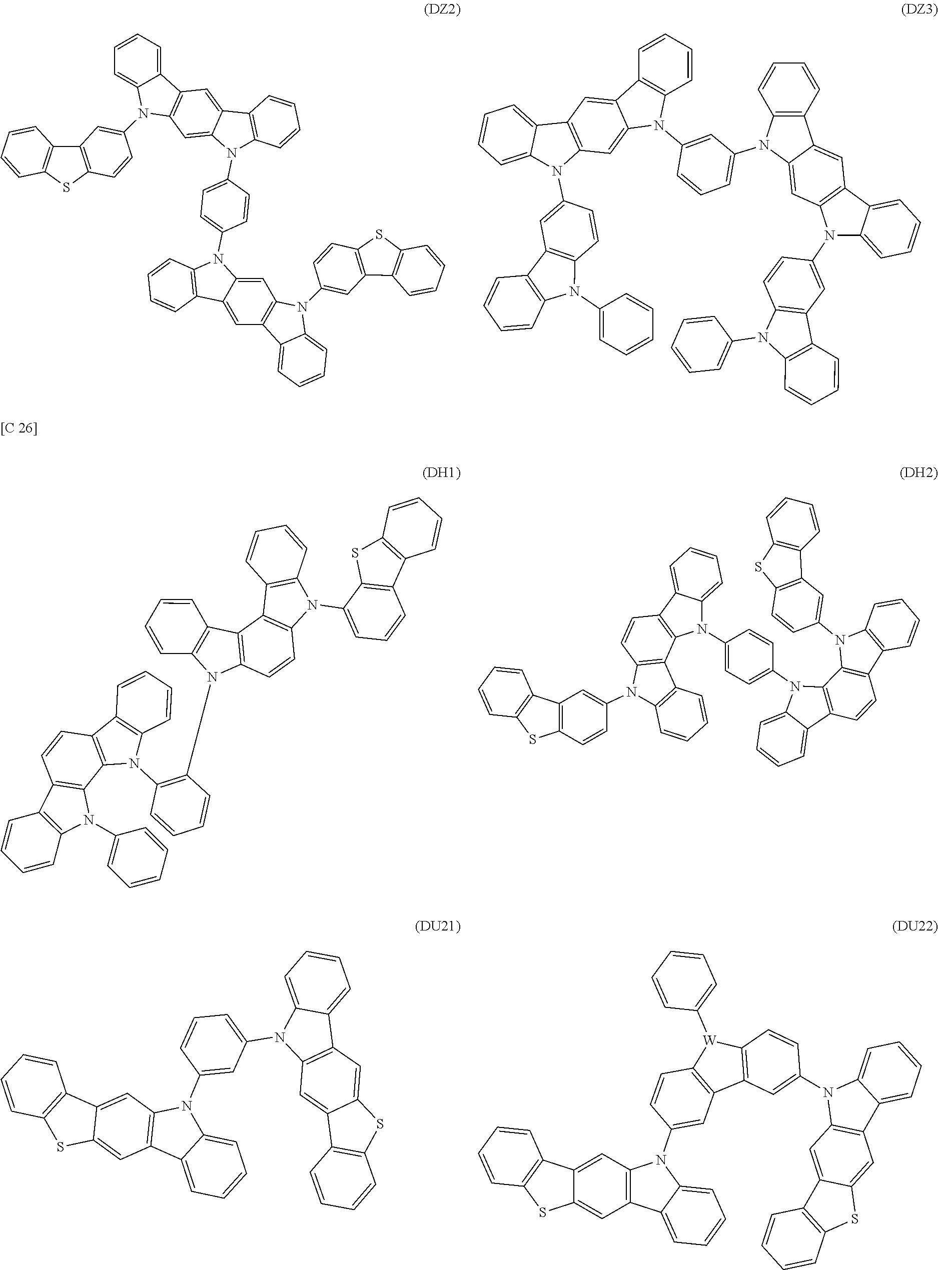

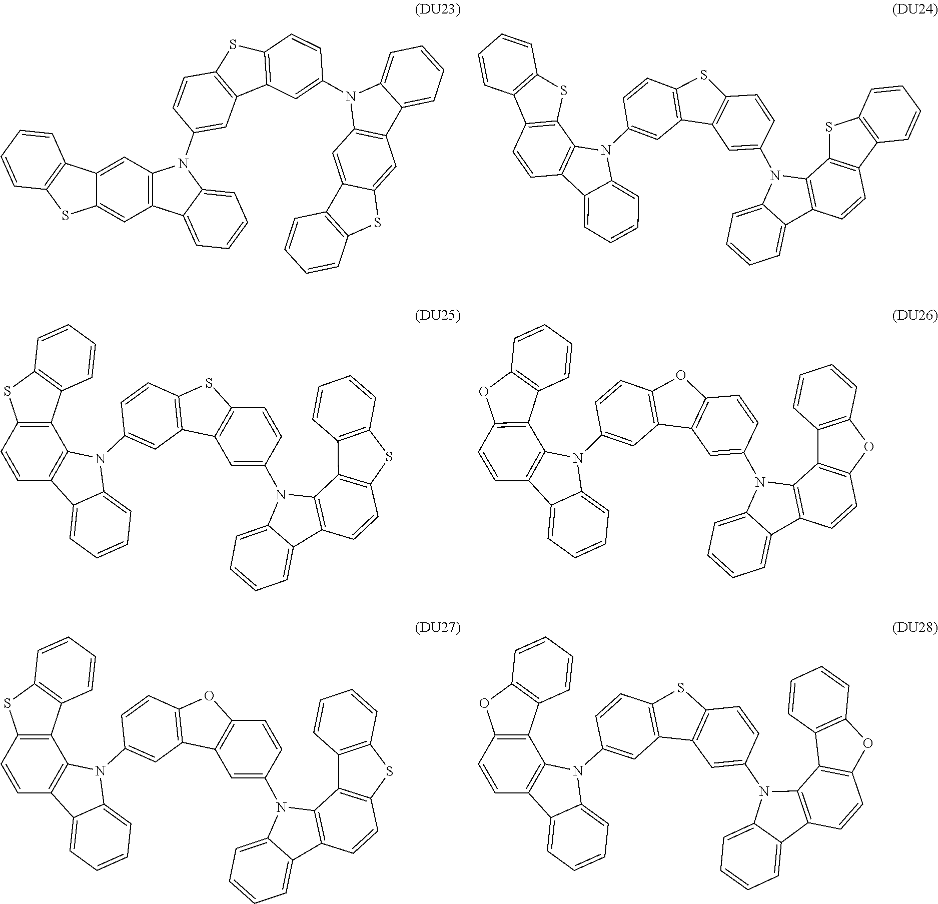

The material for the photoelectric conversion element represented by the general formulae (1) and (2) can be obtained by: synthesis by methods of various organic synthetic reactions established in the field of the organic synthetic chemistry including coupling reactions such as Suzuki coupling, Stille coupling, Grignard coupling, Ullmann coupling, Buchwald-Hartwig reaction, and Heck reaction, using commercially available reagents as raw materials; and then purification by using a known method such as recrystallization, column chromatography, and sublimation and purification. The method is not limited to this method.

The material for a photoelectric conversion element of the present invention preferably has an energy level of HOMO obtained by structural optimization calculation with a density functional calculation B3LYP/6-31G(d) of −4.5 eV or lower, more preferably within a range of −4.5 eV to −6.0 eV.

The material for a photoelectric conversion element for imaging of the present invention preferably has an energy level of LUMO obtained by the above structural optimization calculation of −2.5 eV or higher, more preferably within a range of −2.5 eV to −0.5 eV. In addition, a difference (absolute value) between the HOMO energy level and the LUMO energy level is preferably within a range of 2.0 to 5.0 eV, and more preferably within a range of 2.5 to 4.0 eV.

The material for a photoelectric conversion element of the present invention preferably has a hole mobility of 1×10⁻⁶ cm²/Vs to 1 cm²/Vs, more preferably has a hole mobility of 1×10⁻⁵ cm²/Vs to 1 cm²/Vs. The hole mobility can be evaluated by known methods such as a method with a FET-type transistor element, a method with a time-of-flight method, and an SCLC method.

The material for a photoelectric conversion element of the present invention is preferably amorphous. The amorphousness can be confirmed by various methods, and can be confirmed by, for example, detecting no peak in an XRD method or by detecting no endothermic peak in a DSC method.

Next, a photoelectric conversion element for imaging using the material for a photoelectric conversion element of the present invention will be described with reference to Drawing, but a structure of the photoelectric conversion element of the present invention is not limited thereto.

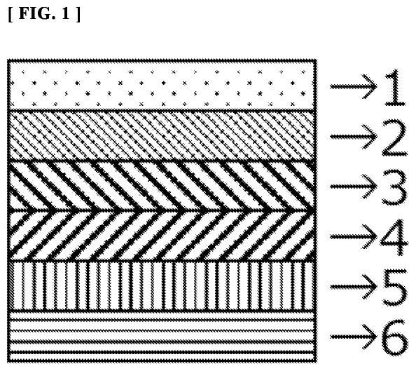

FIG. 1 is a sectional view schematically illustrating a structural example of the photoelectric conversion element for imaging of the present invention.

In FIG. 1, 1 represents a substrate, 2 represents an electrode, 3 represents an electron blocking layer, 4 represents a photoelectric conversion layer, 5 represents a hole blocking layer, and 6 represents an electrode. The photoelectric conversion element is not limited to the structure in FIG. 1, and adding or omitting a layer can be made as necessary.

The material for a photoelectric conversion element of the present invention can be used as an electron transport material. In this case, this material can be used for the photoelectric conversion layer or the hole blocking layer.

Hereinafter, each member and each layer of the photoelectric conversion element of the present invention will be described.

Substrate

The photoelectric conversion element using the material for a photoelectric conversion element of the present invention is preferably supported on a substrate. The substrate is not particularly limited, and substrates made of glass, transparent plastic, quartz, and the like can be used, for example.

Electrode

An electrode used for the photoelectric conversion element for imaging has a function of trapping a hole and an electrode generated in the photoelectric conversion layer. A function to let light enter the photoelectric conversion layer is also required. Thus, at least one of two electrodes is desirably transparent or semi-transparent. A material used for the electrode is not particularly limited as long as it has conductivity, and examples thereof include: conductive transparent materials, such as ITO, IZO, SnO₂, ATO (antimony-doped tin oxide), ZnO, AZO (Al-doped zinc oxide), GZO (gallium-doped zinc oxide), TiO₂, and FTO; metals, such as gold, silver, platinum, chromium, aluminum, iron, cobalt, nickel, and tungsten; inorganic conductive substances, such as copper iodide and copper sulfide; and conductive polymers, such as polythiophene, polypyrrole, and polyaniline. A plurality of these materials may be mixed to use as necessary, and two or more layers thereof may be stacked.

Photoelectric Conversion Layer

The photoelectric conversion layer is a layer in which a hole and an electrode are generated by charge separation of an exciter generated by the incident light. The photoelectric conversion layer may be formed with a single photoelectric converting material, or may be formed by combination with a P-type organic semiconductor material being a hole transport material and an N-type organic semiconductor material being an electron transport material. Two or more kinds of the P-type organic semiconductor may be used, and two or more kinds of the N-type organic semiconductor may be used. One or more kinds of these P-type organic semiconductor and/or N-type organic semiconductor desirably use a dye material having a function of absorbing light with a desired wavelength in the visible region. As the P-type organic semiconductor material being the hole transport material, the material for the photoelectric conversion element represented by the formula (1) or (2) can be used.

The P-type organic semiconductor material may be any material having a hole transportability. The material of the present invention represented by the formula (1) or the general formula (2) is preferably used, but another P-type organic semiconductor material may be used. In addition, two or more kinds of the material represented by the formula (1) or the general formula (2) may be mixed to use. Furthermore, the material of the present invention and another P-type organic semiconductor material may be mixed to use. The another P-type organic semiconductor material may be any material having the hole transportability, and for example, usable are: compounds having a fused polycyclic aromatic group such as naphthalene, anthracene, phenanthrene, pyrene, chrysene, naphthacene, triphenylene, perylene, fluoranthene, fluorene, and indene; compounds having a π-electron excess aromatic group such as a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, a carbazole derivative, and an indolocarbazole derivative; an aromatic amine derivative, a styrylamine derivative, a benzidine derivative, a porphyrin derivative, a phthalocyanine derivative, and a quinacridone derivative.

In addition, examples of a polymer P-type organic semiconductor material include a polyphenylene-vinylene derivative, a polyparaphenylene derivative, a polyfluorene derivative, a polyvinylcarbazole derivative, and a polythiophene derivative. The material of the present invention or a non-polymer P-type organic semiconductor material may be mixed in addition to the polymer P-type organic semiconductor material. Two or more kinds of the polymer P-type organic semiconductor materials may be mixed to use.

The N-type organic semiconductor material may be any material having the electron transportability, and examples thereof include naphthalenetetracarboxylic diimide and perylenetetracarboxylic diimide, fullerenes, and azole derivatives such as imidazole, thiazole, thiadiazole, oxazole, oxadiazole, and triazole. Two or more kinds selected from the N-type organic semiconductor materials may be mixed to use.

Electron Blocking Layer

The electron blocking layer is provided in order to inhibit a dark current generated by injecting an electron from one electrode into the photoelectric conversion layer when a bias voltage is applied between the two electrodes. The electron blocking layer also has a function of hole transportation for transporting a hole generated by charge separation in the photoelectric conversion layer toward the electrode. A single layer or multiple layers of the electron blocking layer can be disposed as necessary. For the electron blocking layer, a P-type organic semiconductor material being the hole transport material can be used. The P-type organic semiconductor material may be any material having the hole transportability. Although the material of the present invention is preferably used, another P-type organic semiconductor material may be used. The material represented by the general formula (1) and the material represented by the general formula (2) may be mixed to use. Furthermore, the material of the present invention and another P-type organic semiconductor material may be mixed to use. The other P-type organic semiconductor material may be any material having the hole transportability, and for example, usable are: compounds having a fused polycyclic aromatic group such as naphthalene, anthracene, phenanthrene, pyrene, chrysene, naphthacene, triphenylene, perylene, fluoranthene, fluorene, and indene; compounds having a π-electron excess aromatic group such as a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, and a carbazole derivative; an aromatic amine derivative, a styrylamine derivative, a benzidine derivative, a porphyrin derivative, a phthalocyanine derivative, and a quinacridone derivative.

Hole Blocking Layer

The hole blocking layer is provided in order to inhibit a dark current generated by injecting a hole from one electrode into the photoelectric conversion layer when a bias voltage is applied between the two electrodes. The hole blocking layer also has a function of electron transportation for transporting an electron generated by charge separation in the photoelectric conversion layer toward the electrode. A single layer or multiple layers of the hole blocking layer can be disposed as necessary. For the hole blocking layer, the N-type organic semiconductor material having the electron transportability can be used. The N-type organic semiconductor material may be any material having the electron transportability, and examples thereof include: polycyclic aromatic multivalent carboxylic anhydride or imidized products thereof, such as naphthalenetetracarboxylic diimide and perylenetetracarboxylic diimide; fullerenes, such as C60 and C70; azole derivatives, such as imidazole, thiazole, thiadiazole, oxazole, oxadiazole, and triazole; a tris(8-quinolinolate)aluminum (III) derivative, a phosphine oxide derivative, a nitro-substituted fluorene derivative, a diphenylquinone derivative, a thiopyran dioxide derivative, a carbodiimide, a fluorenylidene methane derivative, an anthraquinodimethane derivative and an anthrone derivative, a bipyridine derivative, a quinoline derivative, and an indolocarbazole derivative. Two or more kinds selected from the N-type organic semiconductor materials may be mixed to use.

A method for producing a film of each layer in producing the photoelectric conversion element for imaging of the present invention is not particularly limited. The photoelectric conversion element may be produced by any one of dry process and wet process.

### EXAMPLES

Hereinafter, the present invention will be described in more detail with Examples, but the present invention is not limited to these Examples.

Calculation Example (Calculation of HOMO and LUMO Values)

Calculated were HOMO and LUMO of the compound V1 and compounds shown in Table 1. The calculation was performed by using a density functional theory (DFT), using Gaussian as a calculation program, and with the B3LYP/6-31G(d). Table 1 shows the results.

It can be mentioned that any of the materials for the photoelectric conversion element of the present invention has preferable HOMO and LUMO values.

TABLE 1

| | HOMO | LUMO |
|---|---|---|
| Compound | [eV] | [eV] |
| V1 | −5.0 | −1.2 |
| V2 | −4.9 | −1.2 |
| V3 | −4.9 | −1.3 |
| V4 | −5.0 | −1.1 |
| V5 | −4.9 | −1.0 |
| V6 | −4.9 | −1.0 |
| V7 | −4.9 | −0.9 |
| W1 | −5.0 | −0.9 |
| W26 | −5.1 | −0.8 |
| W42 | −4.9 | −1.1 |
| Y1 | −4.9 | −1.5 |
| Y2 | −4.7 | −1.0 |
| Y3 | −4.7 | −1.0 |
| Z1 | −5.0 | −1.2 |
| Z4 | −4.8 | −1.0 |
| U101 | −5.2 | −1.0 |
| U102 | −5.1 | −1.1 |
| U103 | −5.1 | −1.1 |
| U201 | −5.2 | −1.0 |
| U202 | −5.2 | −1.3 |
| DV1 | −5.1 | −0.8 |
| DV2 | −5.0 | −1.4 |
| DW1 | −5.1 | −1.1 |
| DX1 | −4.9 | −2.1 |
| DH1 | −4.8 | −1.1 |

### Synthesis Example 1

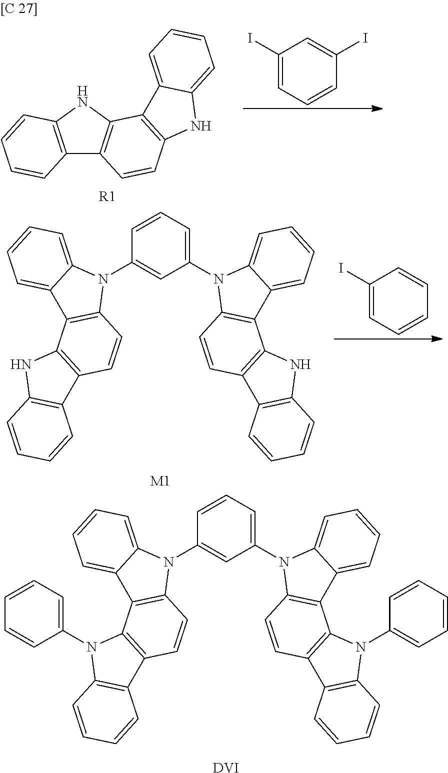

At a room temperature under a nitrogen atmosphere, a raw material R1 (27.3 mmol), 1,3-diiodobenzene (13.6 mmol), tripotassium phosphate (110.7 mmol), and 1,2-cyclohexanediamine (9.6 mmol) were added into dioxane (100 ml), and the mixture was stirred at 100° C. for 3 hours. The mixture was cooled to a room temperature, and then the insoluble product was filtered off and the filtrate was condensed. The condensed residue was added into distilled water (100 ml), and the mixture was stirred at a room temperature. After 3 hours, the precipitate was filtered and then dried to obtain an intermediate M1. The yield was 87%.

At a room temperature under a nitrogen atmosphere, the intermediate M1 (11.9 mmol), potassium carbonate (66.6 mmol), and CuI (38.1 mmol) were added into iodobenzene (50 ml), and the mixture was stirred for 8 hours with reflux heating. The mixture was cooled to a room temperature, the insoluble product was then filtered off, and the filtrate was added into methanol (100 ml), and stirred at a room temperature. After 3 hours, the precipitate was filtered. The obtained crude product was washed with meta-xylene to obtain a target compound DV1 as a yellow solid. The yield was 69%. The obtained powder was evaluated by an XRD method but no peak was detected. Thus, this compound was found to be amorphous.

### Synthesis Example 2

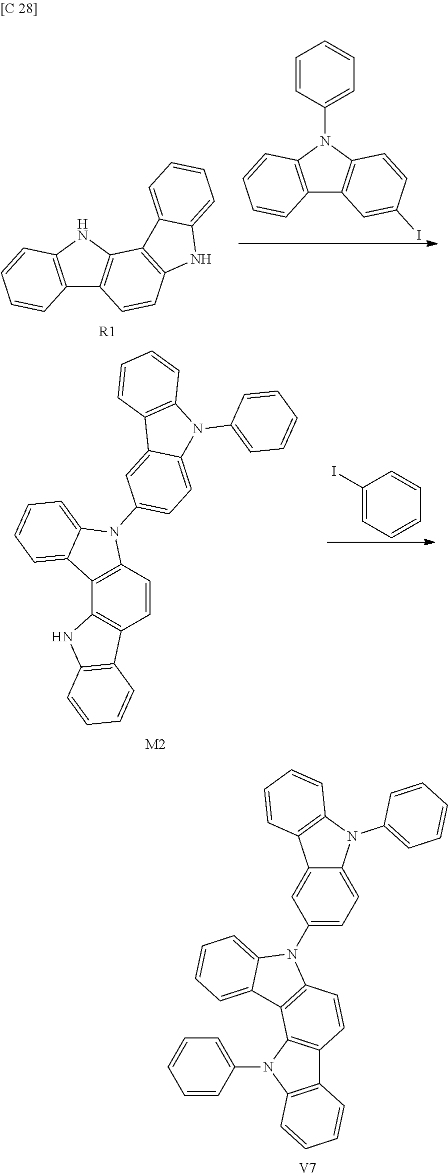

At a room temperature under a nitrogen atmosphere, the raw material R1 (15.6 mmol), 3-iodo-9-phenylcarbazole (15.6 mmol), CuI (1.6 mmol), tripotassium phosphate (62.4 mmol), and 1,2-cyclohexanediamine (14.8 mmol) were added into dioxane (100 ml), and the mixture was stirred at 100° C. for 5 hours. The mixture was cooled to a room temperature, and then the insoluble product was filtered off and the filtrate was condensed. The condensed residue was subjected to silica gel column chromatography (methylene chloride; hexane) to obtain an intermediate M2. The yield was 42%.

At a room temperature under a nitrogen atmosphere, the intermediate M2 (6.5 mmol), copper powder (16.1 mmol), and potassium carbonate (35.5 mmol) were added into iodobenzene (50 ml), and the mixture was stirred for 22 hours with reflux heating. The mixture was cooled to a room temperature and then condensed under a reduced pressure, and the obtained condensed residue was subjected to silica gel column chromatography (methylene chloride; hexane) to obtain a target compound V7 as a white solid. The yield was 81%. The obtained powder was evaluated by an XRD method and found to be amorphous.

### Synthesis Example 3

A target compound V5 was obtained as a white solid in the same procedure as in Synthesis Example 2 except that 2-iodo-9-phenylcarbazole was used instead of 3-iodo-9-phenylcarbazole. The yield was 35%. The obtained powder was evaluated by an XRD method and found to be amorphous.

### Synthesis Example 4

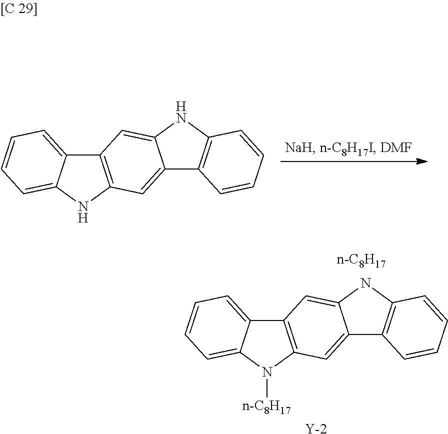

At a room temperature under a nitrogen atmosphere, sodium hydride (150 mmol) was added into a DMF solution (500 ml) of 3,2b-indolocarbazole (50 mmol), and the mixture was stirred at a room temperature. After 30 minutes, 1-iodooctane (133 mmol) was added dropwise thereinto at the same temperature over 30 minutes. The mixture was stirred for 2 hours, and then the reaction liquid was added dropwise to distilled water (1000 ml). The precipitate to be formed was filtered and then dried to obtain a crude product. The obtained crude product was purified by recrystallization (isopropyl alcohol: hexane) to obtain a target compound Y2 as a yellow solid. The yield was 66%. The obtained yellow solid was evaluated by an XRD method and found to be amorphous.

### Synthesis Example 5

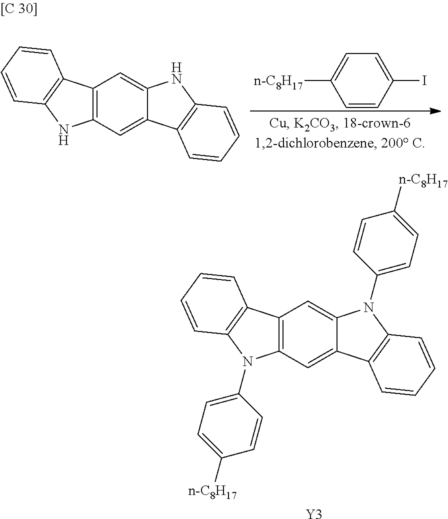

At a room temperature under a nitrogen atmosphere, a 1,2-dichlorobenzene solution (50 ml) of 3,2b-indolocarbazole (12.1 mmol), 48.4 mmol of copper powder, anhydrous potassium carbonate (96.8 mmol), 18-crown-6 (2.42 mmol), and 4-iodooctylbenzene (36.3 mmol) was stirred at 200° C. under a nitrogen atmosphere. After 24 hours, at a room temperature, tetrahydrofuran (150 ml) was added, then the mixture was filtered and the obtained mother liquid was condensed. Methanol (300 ml) was added into the condensed residue, and the generated precipitate was filtered and then dried to obtain a crude product. The obtained crude product was purified by recrystallization (isopropyl alcohol: hexane) to obtain a target compound Y3 as a yellow solid. The yield was 63%. The obtained powder was evaluated by an XRD method and found to be amorphous.

Prepared was a sample in which a layer of the compound DV1 with approximately 3 μm in film thickness was formed between a transparent electrode composed of ITO and an aluminum electrode. The hole mobility was measured by a time-of-flight apparatus (method). The hole mobility was 2×10⁻⁴ cm²/Vs.

Hole mobilities of compounds shown in Table 2 were evaluated in the same manner as above. Table 2 shows the results.

TABLE 2

| | Hole mobility |
|---|---|
| Compound | [cm²/Vs] |
| V1 | 2 × 10⁻⁴ |
| V2 | 1 × 10⁻⁵ |
| V3 | 2 × 10⁻⁵ |
| V4 | 2 × 10⁻⁵ |
| V5 | 1 × 10⁻⁴ |
| V7 | 4 × 10⁻⁵ |
| W1 | 3 × 10⁻⁵ |
| W26 | 1 × 10⁻⁴ |
| W42 | 1 × 10⁻⁴ |
| Y2 | 1 × 10⁻³ |
| Y3 | 5 × 10⁻² |
| Z4 | 6 × 10⁻⁵ |
| DV1 | 2 × 10⁻⁴ |
| DV2 | 1 × 10⁻⁴ |
| DW1 | 4 × 10⁻⁴ |

### Example 1

On a glass substrate on which an electrode composed of ITO with 70 nm in film thickness was formed, a film of the compound DV1 was formed with 100 nm in thickness with a vacuum degree of 4.0×10⁻⁵ Pa as an electron blocking layer. Then, a thin film of quinacridone was formed with 100 nm in thickness as a photoelectric conversion layer. Finally, an aluminum film was formed with 70 nm in thickness was formed as an electrode to produce a photoelectric conversion element.

A current in a dark place was 7.8×10⁻¹² A/cm² with the electrodes of ITO and aluminum and with applying a voltage of 2 V. When a voltage of 2 V was applied and the ITO electrode side was irradiated with light with an LED adjusted to be a irradiation light wavelength of 500 nm and 1.6 μW from a height of 10 cm, a current was 3.1×10⁻⁶ A/cm². A contrast ratio was 3.9×10⁵ with applying a voltage of 2 V on the transparent conductive glass side.

### Comparative Example 1

On a glass substrate on which an electrode composed of ITO with 70 nm in film thickness was formed, a film of quinacridone was formed with 100 nm in thickness with a vacuum degree of 4.0×10⁻⁵ Pa as a photoelectric conversion layer. Finally, an aluminum film was formed with 70 nm in thickness as an electrode to produce a photoelectric conversion element. A current in a dark place was 6.3×10⁻⁸ A/cm² with the electrodes of ITO and aluminum and with applying a voltage of 2 V. When a voltage of 2 V was applied and the ITO electrode side was irradiated with light with an LED adjusted to be an irradiation light wavelength of 500 nm and 1.6 μW from a height of 10 cm, a current was 8.6×10⁻⁶ A/cm². A contrast ratio was 1.4×10² with applying a voltage of 2 V.

### Example 2

On a glass substrate on which an electrode composed of ITO with 70 nm in film thickness was formed, a 10-nm film of the compound W1 was formed with a vacuum degree of 4.0×10⁻⁵ Pa as an electron blocking layer. Then, 2Ph-BTBT, F6-SubPc-OC6F5, and fullerene (C60) were co-deposited at a deposition rate ratio of 4:4:2 with 200 nm to form a film. Subsequently, 10-nm of dpy-NDI was deposited to form a hole blocking layer. Finally, an aluminum film was formed with 70 nm in thickness as an electrode to produce a photoelectric conversion element. A current in a dark place (dark current) was 6.3×10⁻¹⁰ A/cm² with the electrodes of ITO and aluminum and with applying a voltage of 2.6 V. When a voltage of 2.6 V was applied and the ITO electrode side was irradiated with light with an LED adjusted to be a irradiation light wavelength of 500 nm and 1.6 μW from a height of 10 cm, a current (bright current) was 3.0×10⁻⁷ A/cm². A contrast ratio was 4.8×10² with applying a voltage of 2.6 V.

### Examples 3 to 6

Photoelectric conversion elements were produced in the same manner as in Example 2 except that compounds shown in Table 3 were used for the electron blocking layer.

### Comparative Example 2

A photoelectric conversion element was produced in the same manner as in Example 2 except that CzBDF was used for the electron blocking layer.

Table 3 shows the results of Examples and Comparative Examples.

The compounds used in Examples and Comparative Examples are shown below.

TABLE 3
[C 31]

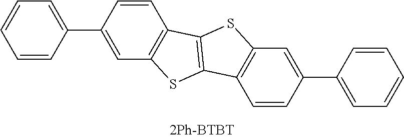 [SMILES: c1ccc(-c2ccc3c(c2)sc2c4ccc(-c5ccccc5)cc4sc32)cc1]

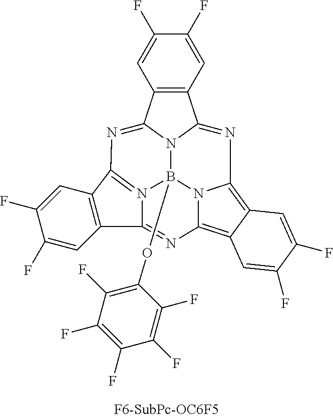

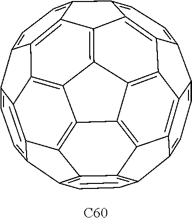 [SMILES: c1cc2c3ccc4c5ccc6c7ccc8c9ccc%10c1c1c2c2c3c4c3c5c6c4c7c8c5c9c%10c1c1c2c3c4c51 ; "C60"]

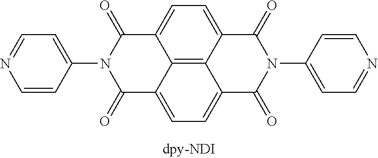 [SMILES: O=C1c2ccc3c4c(ccc(c24)C(=O)N1c1ccncc1)C(=O)N(c1ccncc1)C3=O]

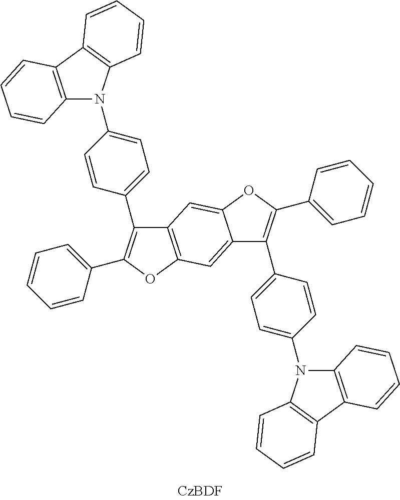 [SMILES: c1ccc(-c2oc3cc4c(-c5ccc(-n6c7ccccc7c7ccccc76)cc5)c(-c5ccccc5)oc4cc3c2-c2ccc(-n3c4ccccc4c4ccccc43)cc2)cc1]

| | | Dark current | Bright current | |
|---|---|---|---|---|
| | Compound | [A/cm²] | [A/cm²] | Contrast ratio |
| Example 2 | W1 | 6.3 × 10⁻¹⁰ | 3.0 × 10⁻⁷ | 4.8 × 10² |
| Example 3 | V7 | 7.8 × 10⁻¹⁰ | 2.6 × 10⁻⁷ | 3.3 × 10² |
| Example 4 | Z4 | 7.2 × 10⁻¹⁰ | 2.5 × 10⁻⁷ | 3.5 × 10² |
| Example 5 | W26 | 5.5 × 10⁻¹⁰ | 3.2 × 10⁻⁷ | 5.8 × 10² |
| Example 6 | W42 | 6.4 × 10⁻¹⁰ | 3.3 × 10⁻⁷ | 5.2 × 10² |
| Comparative | CzBDF | 1.6 × 10⁻⁹ | 1.4 × 10⁻⁷ | 8.4 × 10¹ |
| Example 1 | | | | |

### REFERENCE SIGNS LIST

1 Electrode

2 Hole blocking layer

3 Photoelectric conversion layer

4 Electron blocking layer

5 Electrode

6 Substrate

1. A material for a photoelectric conversion element for imaging represented by the following general formula (1) or (2), 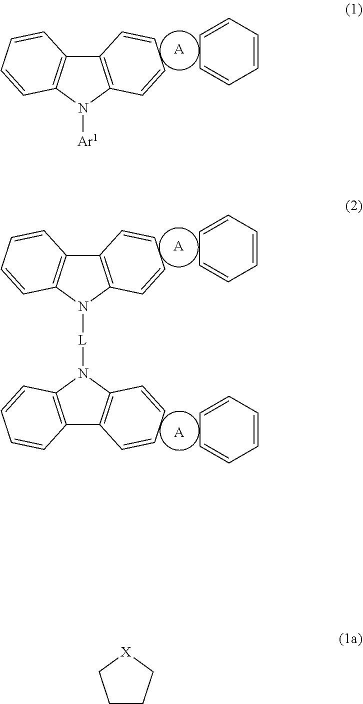
wherein in the general formulae (1) and (2), ring A independently represents a heterocyclic ring represented by the formula (1a) and fused with an adjacent ring at any position;
X represents O, S, or N—Ar²;
Ar¹ and Ar² each independently represent an alkyl group having 1 to 20 carbon atoms, a substituted or non-substituted aromatic hydrocarbon group having 6 to 30 carbon atoms, a substituted or non-substituted π-electron excess heteroaromatic group having 4 to 30 carbon atoms, or a substituted or non-substituted linked aromatic group formed by linking of two to six aromatic rings of aromatic groups selected from the aromatic hydrocarbon group and the π-electron excess heteroaromatic group; provided that, when Ar¹ and Ar² are linked aromatic groups formed only by the aromatic hydrocarbon group, Ar¹ and Ar² are not simultaneously biphenyl groups; and
L represents a divalent substituted or non-substituted aromatic hydrocarbon group having 6 to 30 carbon atoms, a substituted or non-substituted π-electron excess heteroaromatic group having 4 to 30 carbon atoms, or a substituted or non-substituted linked aromatic group formed by linking of two to six aromatic rings of aromatic groups selected from the aromatic hydrocarbon group and the π-electron excess heteroaromatic group.

2. The material for a photoelectric conversion element for imaging according to claim 1, wherein at least one of the Ar¹ and Ar² contains at least one substituted or non-substituted tricyclic fused skeleton.

3. The material for a photoelectric conversion element for imaging according to claim 2, wherein the tricyclic fused skeleton is at least one selected from a carbazole, dibenzofuran, or dibenzothiophene skeleton.

4. The material for a photoelectric conversion element for imaging according to claim 2, wherein the tricyclic fused skeleton is a carbazole skeleton.

5. The material for a photoelectric conversion element for imaging according to claim 1, wherein an energy level of highest occupied molecular orbital (HOMO) obtained by structural optimization calculation with a density functional calculation B3LYP/6-31G(d) is −4.5 eV or lower.

6. The material for a photoelectric conversion element for imaging according to claim 1, wherein an energy level of a lowest unoccupied molecular orbital (LUMO) obtained by structural optimization calculation with a density functional calculation B3LYP/6-31G(d) is −2.5 eV or higher.

7. The material for a photoelectric conversion element for imaging according to claim 1 wherein the material has a hole mobility of 1×10⁻⁶ cm²/Vs or more.

8. The material for a photoelectric conversion element for imaging according to claim 1, wherein the material is amorphous.

9. The material for a photoelectric conversion element for imaging according to claim 1 wherein the material is used as a hole transport material of a photoelectric conversion element for imaging.

10. A photoelectric conversion element for imaging, comprising a photoelectric conversion layer and an electron blocking layer between two electrodes, wherein at least one layer of the photoelectric conversion layer and the electron blocking layer contains the material for a photoelectric conversion element for imaging according to claim 1.

11. The photoelectric conversion element for imaging according to claim wherein the photoelectric conversion layer contains an electron transport material.

12. The photoelectric conversion element for imaging according to claim 10, wherein the electron blocking layer contains the material for a photoelectric conversion element for imaging.
